(19) United States
(12) Patent Application Publication
(10) Pub. No.: US 2026/0363666 A1
(43) Pub. Date: Nov. 19, 2026

(54) INTRAVASCULAR IMAGE PROCESSING FOR ATTENUATED PLAQUE DETECTION

(71) Applicant: Boston Scientific Scimed, Inc., Maple Grove, MN (US)

(72) Inventors: Yan Li, Plymouth, MN (US); Kevin Bloms, Minneapolis, MN (US); Qian Li, Saratoga, CA (US)

(73) Assignee: Boston Scientific Scimed, Inc., Maple Grove, MN (US)

(21) Appl. No.: 19/678,930

(22) Filed: May 15, 2026

Related U.S. Application Data
(60) Provisional application No. 63/806,494, filed on May 15, 2025.

(51) Int. Cl. A61B 8/00; A61B 8/08; A61B 8/12; G06F 3/04847; G06F 3/0485

(52) U.S. Cl. CPC A61B 8/465; A61B 8/0891; A61B 8/12; A61B 8/463; A61B 8/469; G06F 3/04847; G06F 3/0485

(57) ABSTRACT

The present disclosure provides a graphical user interface (GUI) arranged to convey information related to the IVUS images and attenuated plaque detected in a vessel of a patient, such as an artery, as represented in the IVUS images. The GUIs can be generated to include a cross-section view and a longitudinal view of the vessel and an indication of locations of attenuated plaque relative to a frame depicted in the cross-sectional view. The GUIs may also be generated to provide various graphical depictions of a mapping of detected attenuated plaque positioned with an imaged portion of a vessel of a patient.

### CROSS-REFERENCE TO RELATED APPLICATION

This application claims the benefit of U.S. Patent Application Serial No. 63/806,494, filed May. 15, 2025, entitled " INTRAVASCULAR IMAGE PROCESSING FOR ATTENUATED PLAQUE DETECTION”, which is incorporated by reference herein in its entirety.

### TECHNICAL FIELD

The present disclosure generally relates to intravascular imaging systems, such as intravascular ultrasound (IVUS). Particularly, but not exclusively, the present disclosure relates to an improved image processing for use in the detection of attenuated plaque in a vessel of a patient.

### BACKGROUND

Intravascular imaging (IVI) devices are insertable into a patient's vasculature and configured to capture images from within the vessel lumen. Two common intravascular imaging modalities are intravascular ultrasound (IVUS) and optical coherence tomography (OCT). Such imaging modalities have proven diagnostic capabilities for a variety of diseases and disorders. For example, IVI devices are often used as part of a percutaneous coronary intervention (PCI) and can be used to plan, treat, and assess treatment for various obstructive coronary artery diseases, such as, unstable angina, acute myocardial infarction (MI), coronary artery disease (CAD), or the like.

As noted, an example IVI system is an IVUS system. An IVUS system includes a control module with a pulse generator, a motor drive unit, image acquisition and processing components, and a monitor. The IVUS system further includes a catheter with an ultrasound transducer included as part of the distal end of the catheter. The catheter is positioned in a lumen or cavity within, or in proximity to, a region to be imaged, such as a cardiac vessel. Often, a series of images or series of image frames are captured while the catheter is moved (e.g., pulled proximally, or the like) within the vessel lumen.

This series of image frames represent the vessel lumen structure (e.g., vessel wall, lumen, plaque, calcium, stents, etc.) along a longitudinal section of the vessel. Such images can be captured as part of a pre-PCI procedure to aid a physician in determining how to treat the patient, for example, what stent size is appropriate to treat a stenosis, stent landing zones, or the like. Further, such images can be captured as part of a post-PCI procedure to assess the results of the procedure, such as the placement and/or expansion of the stent.

However, it can be difficult for physicians to visualize the complete structure of the vessel lumen and/or the effectiveness of the PCI from the raw series of image frames, in particular with respect to the presence of attenuated plaque. For example, with current IVI systems, the physician must attempt to identify areas where attenuated plaque resides with a vessel of a patient manually based on reviewing the captured images of the vessel. The presence of attenuated plaque in the vessel may present an increased risk of events like a rupture, which can then result in a sudden blockage of the artery where or downstream from where the rupture has occurred, leading to an acute coronary event such as a heart attack. Attenuated plaque is difficult to discern in a typical IVI image. Absorption and scattering of the ultrasound signal due to the presence of attenuated plaque results in a low level of echo return in the imaging in the areas of the vessel being imaged using IVUS imaging system and techniques. The physician may use this information to make their clinical decisions. However, as this process is manual, it increases procedure lengths and is a potential area where misinterpretation of the presence and/or of the extent and precise location(s) of attenuated plaque within the vessel can occur. Thus, there is a need for an improved way to provide detection and indications of attenuated plaque.

### BRIEF SUMMARY

This Brief Summary is provided to introduce a selection of concepts in a simplified form that are further described below in the Detailed Description. This Summary is not intended to necessarily identify key features or essential features of the claimed subject matter, nor is it intended as an aid in determining the scope of the claimed subject matter.

In general, the present disclosure provides systems and techniques to detect attenuated plaque from a series of image frames captured via an IVI modality and to generate a graphical user interface (GUI) providing graphical indications of location within a vessel of a patient where attenuated plaque is indicated.

For example, the present disclosure provides an improvement to computing devices and particularly to IVI guidance systems (e.g., an IVUS guidance system, or the like) in that the IVI guidance system provides a graphical user interface arranged to convey the indicated presence of attenuated plaque and locations of the attenuated plaque along the imaged length of the vessel. The examples described herein use an IVUS guidance system as the basis of the examples. However, other IVI modalities could be implemented (e.g., OCT, or the like).

With some embodiments, an IVUS guidance system may include use of various image processing tools to process and analyze the signals generated during an IVUS imaging run. In some embodiments, the processing tools may include machine learning tools and/or use of artificial intelligence (AI) to process and analyze the signals generated during an IVUS run. The various image processing tools, which may or may not include machine learning tools and/or AI, can be configured to include automatic detection of calcium, calcium arc length, areas of calcium fracture, and/or calcium reverb within a vessel, and/or the detection of attenuated plaque. For example, embodiments of the image processing tools used to detect the presence and location(s) of attenuated plaque may include use of a vector based analysis to determine the presence and/or location(s) of attenuated plaque based on the processed IVUS images. The detection and determination of the locations (s) of any attenuated plaque based on the analysis of the signals generated during the IVUS run may or may not be combined image processing procedure performed to detect the presence of calcium and/or the presence of calcium reverberation. The system can be configured to generate a GUI that includes indications of any detected calcium, the presence of calcium reverb and/or the presence and location(s) of attenuated plaque, and display the GUI on a display device. Embodiments include a GUI that provides a mapping of the location(s), the extent, and the arrangement of attenuated plaque along the length of the imaged vessel, thereby relieving the physician of manually trying to identify the calcium, calcium reverb, and the attenuated plaque locations along the vessel.

In some implementations, the present disclosure may be embodied as a method for intravascular image visualization, for example a method comprising: receiving a series of intravascular ultrasound (IVUS) images of a vessel of a patient; generating a longitudinal view of the vessel based on the series of IVUS images; generating a first cross-sectional view of a first one of the series of IVUS images and a second cross-sectional view of a second one of the series of IVUS images; generating a graphical user interface (GUI) comprising the longitudinal view, the first cross-sectional view, the second cross-sectional view, a first user-positionable longitudinal marker, and a second user-positionable longitudinal marker, wherein the first longitudinal marker and the second longitudinal marker are each positionable along a longitudinal axis of the longitudinal view, wherein the first longitudinal marker is positionable independently of the second longitudinal marker, wherein the first cross-sectional view is linked to the first longitudinal marker, and wherein the second cross-sectional view is linked to the second longitudinal marker; updating the first cross-sectional view, responsive to a change in a position of the first longitudinal marker along the longitudinal axis, to depict a cross-sectional image of the vessel corresponding to the position of the first longitudinal marker; updating the second cross-sectional view, responsive to a change in a position of the second longitudinal marker along the longitudinal axis, to depict a cross-sectional image of the vessel corresponding to the position of the second longitudinal marker; and rendering the GUI for display on a display.

Alternatively or additionally, any of the embodiments of the method above can comprise wherein at least one of the first user-positionable longitudinal marker or the second user-positionable longitudinal marker comprises a slider bar positionable along the longitudinal axis of the longitudinal view by user input.

Alternatively or additionally, any of the embodiments of the method above can comprise wherein at least one of the first user-positionable longitudinal marker or the second user-positionable longitudinal marker comprises a bookmark associated with a saved position along the longitudinal axis of the longitudinal view.

Alternatively or additionally, any of the embodiments of the method above can further comprise receiving a user input designating a position along the longitudinal axis of the longitudinal view, and generating the bookmark at the designated position responsive to the user input.

Alternatively or additionally, any of the embodiments of the method above can comprise wherein at least one of the first user-positionable longitudinal marker or the second user-positionable longitudinal marker comprises a draggable pin marker.

Alternatively or additionally, any of the embodiments of the method above can comprise wherein the first user-positionable longitudinal marker comprises a slider bar and the second user-positionable longitudinal marker comprises a bookmark associated with a saved position along the longitudinal axis of the longitudinal view.

Alternatively or additionally, any of the embodiments of the method above can further comprise detecting areas of attenuated plaque within the vessel based on the series of IVUS images, and generating a graphical component comprising an indication of the attenuated plaque, wherein the GUI comprises the graphical component.

Alternatively or additionally, any of the embodiments of the method above can comprise wherein the graphical component comprises a first arc extending around at least a portion of a circumference of the vessel depicted in the first cross-sectional view and a second arc extending around at least a portion of a circumference of the vessel depicted in the second cross-sectional view.

Alternatively or additionally, any of the embodiments of the method above can comprise wherein the first cross-sectional view and the second cross-sectional view are displayed side by side within the GUI.

Alternatively or additionally, any of the embodiments of the method above can further comprise determining a longitudinal distance along the vessel between the position of the first user-positionable longitudinal marker and the position of the second user-positionable longitudinal marker, wherein the GUI further comprises an indication of the longitudinal distance.

Alternatively or additionally, any of the embodiments of the method above can further comprise generating a mapping comprising graphical depictions of locations within the vessel where attenuated plaque was detected, the mapping comprising a long view of the series of IVUS images, wherein a longitudinal axis of the vessel is represented on an x axis of the mapping and a circumference of the vessel is represented on a y axis of the mapping, and wherein the GUI further comprises the mapping positioned offset from and aligned with the longitudinal view so that one or more locations within the vessel where attenuated plaque was detected align with corresponding portions of the longitudinal view.

Alternatively or additionally, any of the embodiments of the method above can further comprise generating a graphical display representing a portion of an anatomical structure within which the vessel resides, and generating a position indicator within the graphical display, the position indicator indicating, within the anatomical structure, a location of the vessel corresponding to one of the position of the first user-positionable longitudinal marker or the position of the second user-positionable longitudinal marker, wherein the GUI further comprises the graphical display and the position indicator.

Alternatively or additionally, some of the embodiments of the method above can comprise wherein the position indicator comprises a dashed line.

Alternatively or additionally, any of the embodiments of the method above can comprise wherein the GUI further comprises a toggle input operable to selectively show or hide the graphical display within the GUI.

Alternatively or additionally, any of the embodiments of the method above can further comprise receiving an input from a scroll speed control of the GUI, and automatically advancing the position of the first user-positionable longitudinal marker along the longitudinal axis at a rate corresponding to the input received from the scroll speed control, wherein the first cross-sectional view and the position indicator are updated as the position of the first user-positionable longitudinal marker advances.

Alternatively or additionally, some of the embodiments of the method above can comprise wherein automatically advancing the position of the first user-positionable longitudinal marker comprises advancing the position of the first user-positionable longitudinal marker independently of the position of the second user-positionable longitudinal marker.

Alternatively or additionally, any of the embodiments of the method above can comprise wherein the anatomical structure comprises a portion of cardiac vasculature, and wherein the graphical display comprises a representation of a plurality of branches of the cardiac vasculature.

In some implementations, the present disclosure may be embodied as an apparatus for visualizing intravascular ultrasound images, the apparatus comprising a processor arranged to be coupled to a non-transitory memory storage device, the non-transitory memory storage device comprising computer readable instructions that, when executed by the processor, cause the apparatus to: receive a series of intravascular ultrasound (IVUS) images of a vessel of a patient; generate a longitudinal view of the vessel based on the series of IVUS images; generate a first cross-sectional view of a first one of the series of IVUS images and a second cross-sectional view of a second one of the series of IVUS images; generate a graphical user interface (GUI) comprising the longitudinal view, the first cross-sectional view, the second cross-sectional view, a first user-positionable longitudinal marker, and a second user-positionable longitudinal marker, wherein the first longitudinal marker and the second longitudinal marker are each positionable along a longitudinal axis of the longitudinal view, wherein the first longitudinal marker is positionable independently of the second longitudinal marker, wherein the first cross-sectional view is linked to the first longitudinal marker, and wherein the second cross-sectional view is linked to the second longitudinal marker; update the first cross-sectional view, responsive to a change in a position of the first longitudinal marker along the longitudinal axis, to depict a cross-sectional image of the vessel corresponding to the position of the first longitudinal marker; update the second cross-sectional view, responsive to a change in a position of the second longitudinal marker along the longitudinal axis, to depict a cross-sectional image of the vessel corresponding to the position of the second longitudinal marker; and render the GUI for display on a display.

Alternatively or additionally, the apparatus of any of the above embodiments may include wherein at least one of the first user-positionable longitudinal marker or the second user-positionable longitudinal marker comprises a slider bar, a bookmark associated with a saved position along the longitudinal axis of the longitudinal view, or a draggable pin marker.

In some implementations, the present disclosure is embodied as a non-transitory computer-readable storage device comprising instructions executable by a processor of a computing device, wherein the instructions, when executed by the processor, cause the computing device to: receive a series of intravascular ultrasound (IVUS) images of a vessel of a patient; generate a longitudinal view of the vessel based on the series of IVUS images; generate a first cross-sectional view of a first one of the series of IVUS images and a second cross-sectional view of a second one of the series of IVUS images; generate a graphical user interface (GUI) comprising the longitudinal view, the first cross-sectional view, the second cross-sectional view, a first user-positionable longitudinal marker, and a second user-positionable longitudinal marker, wherein the first longitudinal marker and the second longitudinal marker are each positionable along a longitudinal axis of the longitudinal view, wherein the first longitudinal marker is positionable independently of the second longitudinal marker, wherein the first cross-sectional view is linked to the first longitudinal marker, and wherein the second cross-sectional view is linked to the second longitudinal marker; update the first cross-sectional view, responsive to a change in a position of the first longitudinal marker along the longitudinal axis, to depict a cross-sectional image of the vessel corresponding to the position of the first longitudinal marker; update the second cross-sectional view, responsive to a change in a position of the second longitudinal marker along the longitudinal axis, to depict a cross-sectional image of the vessel corresponding to the position of the second longitudinal marker; and render the graphical user interface for display on a display.

### BRIEF DESCRIPTION OF THE SEVERAL VIEWS OF THE DRAWINGS

To easily identify the discussion of any element or act, the most significant digit or digits in a reference number refer to the figure number in which that element is first introduced.

FIG. 1A illustrates an example cardiac vasculature structure and an IVUS catheter inserted into the one of the cardiac arteries.

FIG. 1B illustrates an IVUS imaging system including the IVUS catheter shown in FIG. 1A.

FIG. 1C illustrates depictions in cross-sectional views of different types of plaque that may be present in a vessel.

FIG. 2A illustrates an example series of IVUS image frames.

FIG. 2B illustrates a longitudinal representation of the example series of IVUS image frames of FIG. 2A.

FIG. 3 illustrates an IVUS navigation system.

FIG. 4 illustrates a first example graphical user interface (GUI).

FIG. 5A illustrates a second example GUI.

FIG. 5B illustrates a third example GUI.

FIG. 5C illustrates a fourth example GUI.

FIG. 5D illustrates a fifth example GUI.

FIG. 5E illustrates a sixth example GUI.

FIG. 5F illustrates a seventh example GUI.

FIG. 5G illustrates an eight example GUI.

FIG. 6 illustrates a ninth example GUI.

FIG. 7 illustrates a tenth example GUI.

FIG. 8 illustrates an eleventh example GUI.

FIG. 9 illustrates a logic flow for detecting attenuated plaque.

FIG. 10 illustrates a logic flow for mapping attenuated plaque.

FIG. 11 illustrates a computer-readable storage medium.

FIG. 12 illustrates a diagrammatic representation of a machine.

### DETAILED DESCRIPTION

The foregoing has broadly outlined the features and technical advantages of the present disclosure such that the following detailed description of the disclosure may be better understood. It is to be appreciated by those skilled in the art that the embodiments disclosed may be readily utilized as a basis for modifying or designing other structures for carrying out the same purposes of the present disclosure. The novel features of the disclosure, both as to its organization and operation, together with further objects and advantages will be better understood from the following description when considered in connection with the accompanying figures. It is to be expressly understood, however, that each of the figures is provided for the purpose of illustration and description and is not intended as a definition of the limits of the present disclosure.

As noted, the present disclosure relates to IVI guidance systems, such as, IVUS guidance systems. Further, the present disclosure relates to automatic assessment of the IVI images. In particular, the disclosure provides a graphical user interface (GUI) arranged to convey information related to the IVI images and assessment of the vessel and/or lesion, including information related to the presence and location(s) of attenuated plaque within the vessel.

As noted, the disclosure uses IVUS as the example imaging modality. As such, an example IVUS imaging system, cardiac vasculature structure, and series of IVUS images are described prior to describing the improvements to IVI guidance systems and the GUI of the present disclosure.

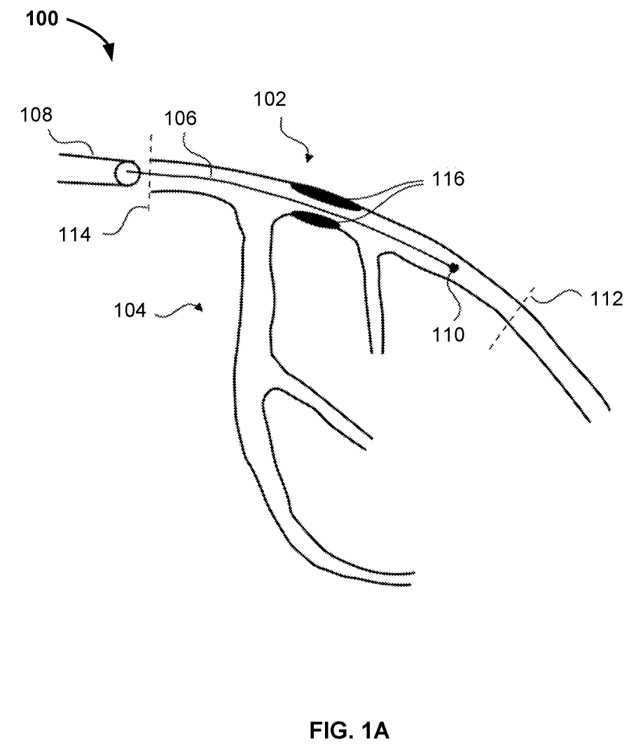

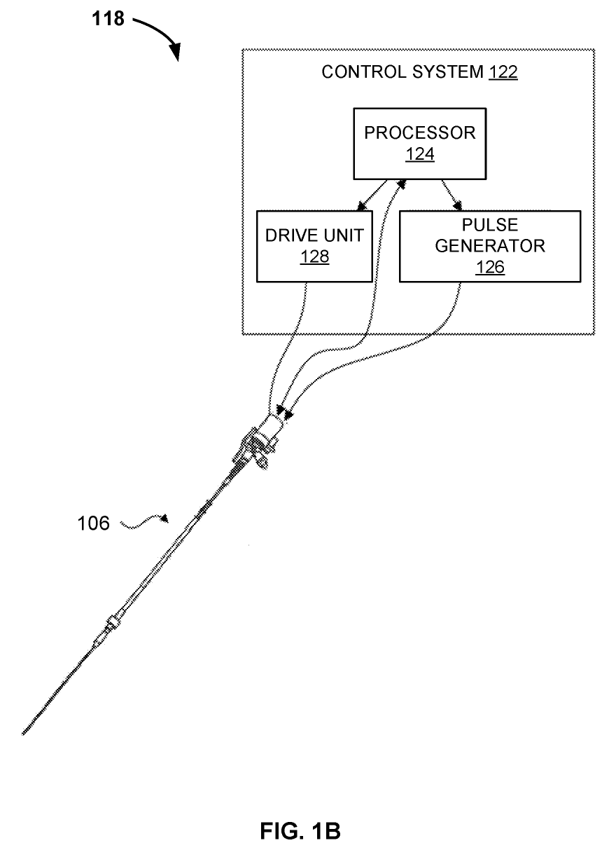

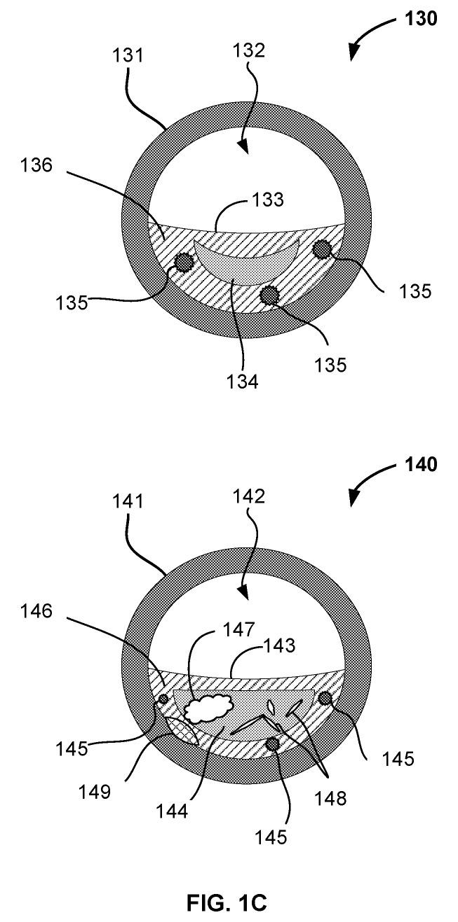

FIG. 1A illustrates a cardiac vasculature structure 100 having a cardiac artery 102 (e.g., cardiac artery, vessel, or the like) with several side branches 104 (e.g., descending arteries, marginal arteries, vessels, or the like) branching off the cardiac artery 102. It is to be appreciated that various cardiac interventions, often referred to as percutaneous coronary intervention (PCI), can be guided by IVUS. For example, prior to performing a PCI procedure, during a PCI procedure, and/or after a PCI procedure; an intravascular imaging catheter (e.g., an IVUS imaging catheter 106) is inserted into the cardiac artery 102 via a guide catheter 108 and images of the cardiac artery 102 are captured. These images are often captured pre-PCI as well as post-PCI. During imaging, the IVUS imaging catheter 106 includes an ultrasound transducer 110 disposed on a distal end of the IVUS imaging catheter 106. The IVUS imaging catheter 106 can be pulled back from a distal point 112 in the cardiac artery 102 to a proximal point 114 in the cardiac artery 102 and intravascular image frames are captured while the IVUS imaging catheter 106 is pulled back.

As indicated in this figure, the cardiac artery 102 includes an obstruction 116 disposed in the vessel, which may comprise calcium and may include attenuated plaque. Often, in a PCI procedure, a physician can choose to break up calcium in the cardiac artery 102 (e.g., the obstruction 116, or the like). It is to be appreciated that by breaking up the calcium or causing a calcium “fracture” the efficacy or outcome of the PCI procedure is improved. For example, fractured calcium allows for better expansion (e.g., stent expansion, or the like). Further, the thickness of the calcium affects calcium fracture treatment selection as well as overall treatment outcomes. However, these procedures need to consider the presence of any attenuated plaque, which could potentially rupture during the fracturing procedure. Therefore, it becomes important for the physician to know about the presence and location(s) of any attenuated plaque included in obstruction 116.

FIG. 1B illustrates an IVUS imaging system 118. The IVUS imaging system 118 is depicted including IVUS imaging catheter 106, which is couplable to a control system 122. The control system 122 may include, for example, processor 124, pulse generator 126, and drive unit 128. In at least some embodiments, the pulse generator 126 forms electric pulses that may be input to one or more transducers (e.g., ultrasound transducer 110, or the like) disposed on IVUS imaging catheter 106.

With some embodiments, ultrasound transducer 110 can be part of an imaging core and coupled to the proximal end of IVUS imaging catheter 106 via a drive cable or drive shaft. Mechanical energy from the drive unit 128 can be used to rotate the imaging core (and thus the ultrasound transducer 110).

The electrical pulses generated by pulse generator 126 can be delivered to the ultrasound transducer 110 while the ultrasound transducer 110 is rotated by the drive unit 128. The electrical signals are transformed by the ultrasound transducer 110 into acoustic pulses that are transmitted through the tissue of cardiac vasculature structure 100. The tissue and/or structure (e.g., calcium, etc.) of the cardiac vasculature structure 100 reflects the acoustic pulses, which are absorbed by the ultrasound transducer 110 and transformed into electric pulses. The transformed electric pulses are delivered to the processor 124 and converted into IVUS images displayable on a monitor. However, as further described below, attenuated plaque may provide a reduced level, or no level at all, of reflection of the acoustic pulses directed at areas within the vessel where the attenuated plaque is located. This lack of reflection of acoustic pulses may present difficulties in determining the presence and/or the extent and location of the attenuated plaque based solely on a manual visual inspection of the resulting ultrasound images.

In general, IVUS imaging system 118 can detect obstruction 116 from images of the cardiac artery 102 captured by the IVUS imaging catheter 106. The calcium, and particularly the locations and calcium reverb, can be identified. For example, processor 124 can implement a calcium detection algorithm. Further, processor 124 can generate a GUI comprising indications of the detected calcium and locations and calcium reverb along the cardiac artery 102. In addition, system, methods, and techniques as further described herein, can detect the presence and location(s) of attenuated plaque within the imaged vessel. The identified locations and the medium identified (e.g., calcium, reverb, attenuated plaque, etc.) can be displayed on a GUI. As such, the GUI provided herein will facilitate quick identification of the presence of calcium, the location(s) of calcium reverb, and the presence and location(s) of any attenuated plaque within the intravascular ultrasound image, thereby increasing workflow, providing improved patient outcomes and patient safety.

FIG. 1C illustrates depictions in cross-sectional views, of different types of plaque that may be present in vessels 130 and 140. The upper depiction in FIG. 1C illustrates a cross-sectional view of a vessel 130 having a vessel wall 131 encircling a lumen 132 extending through the vessel. Vessel 130 includes a portion of the lumen area of the vessel comprising a buildup of stable plaque 136. The stable plaque 136 includes a thick fibrous cap 133 extending across the plaque buildup from one side of the vessel inner wall to an opposite side of the inner wall of the vessel, enclosing a lipid core 134 and a plurality of foam cells 135. While the presence of the stable plaque is less likely to rupture, stable plaque still presents a health risk to the patient as it can reduce or in some instances completely block the flow of the available amount of blood that may be provided through the lumen 132 of the vessel where the plaque resides.

The lower depiction in FIG. 1C illustrates a cross-sectional view of a vessel 140 having a vessel wall 141 encircling a lumen 142 extending through the vessel. Vessel 140 includes a portion of the lumen area of the vessel comprising a buildup of vulnerable plaque 146. The vulnerable plaque 146 includes a thin fibrous cap 143 extending across the plaque buildup from one side of the inner wall of the vessel to an opposite side of the inner wall of the vessel, enclosing a necrotic core 144, a plurality of foam cells 145, intraplaque haemorrahge 147, and calcification 148. As shown in FIG. 1C, vessel 140 may also include attenuated plaque 149.

Unlike stable plaque, which builds up and narrows arteries over time, venerable plaque is more prone to rupture. When rupture occurs, it can cause a sudden blockage in the artery, leading to an acute coronary event. Plaque vulnerability remains a challenge for Target Vessel Failure (TVF) due to untreated/non-culprit lesions – e.g., plaque rupture plus new ischemic lesions. Histopathologically, 91.4% of echo-attenuated plaques correspond to either a fibroatheroma (FA) with a necrotic core (NC) or pathological intimal thickening with a lipid pool. Vessel 140 illustrates a portion for the vulnerable plaque that includes attenuated plaque 148. The ability to detect vulnerable plaque, including attenuated plaque, can allow for treatment that significantly reduces the risk of severe cardiovascular events such as heart attacks and strokes.

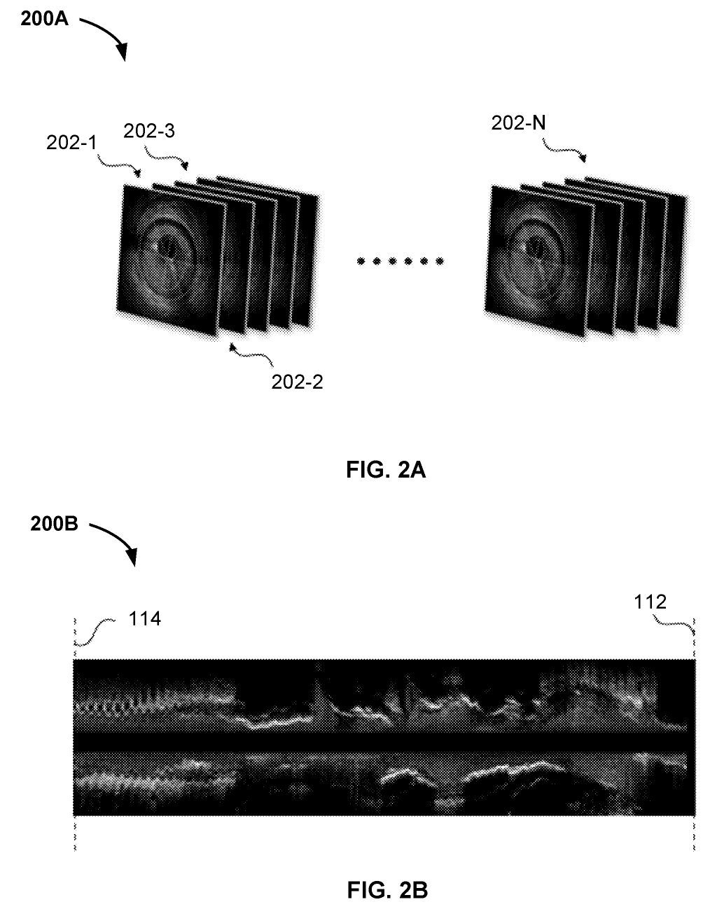

FIGS. 2A and 2B illustrate a series of IVUS image frames 200A and a longitudinal view 200B of the series of IVUS image frames 200A. As described above, several IVUS image frames can be captured while IVUS imaging catheter 106 is moved through cardiac artery 102 (FIG. 1A). For example, FIG. 2A depicts the series of IVUS image frames 200A including IVUS image frames 202-1, 202-2, 202-3 to 202-N, where N is a positive integer, in this case, greater than ten (10). In practice, Ncan be any positive integer greater than or equal to two (2) but will often be greater than one hundred.

The series of IVUS image frames 200A can be stacked or grouped and depicted longitudinally to represent a longitudinal slice of the cardiac artery 102 from distal point 112 to proximal point 114. For example, FIG. 2B illustrates longitudinal view 200B which shows a longitudinal slice of cardiac artery 102 from distal point 112 to proximal point 114 formed from the series of IVUS image frames 200A.

As noted above, calcium and/or calcium reverb may be represented in the series of IVUS image frames 200A. The disclosure provides to detect calcium and calcium reverb from the series of IVUS image frames 200A and to further process these images to detect the presence and location(s) of attenuated plaque based on the IVUS image frames. Embodiments as further disclosed herein include to generate various graphical user interfaces (GUIs) comprising indications of the location of the detected calcium, calcium reverb, and the detected attenuated plaque, including generating and displaying, for example on a display device, graphical depictions illustrating a mapping of the locations of the detected attenuated plaque.

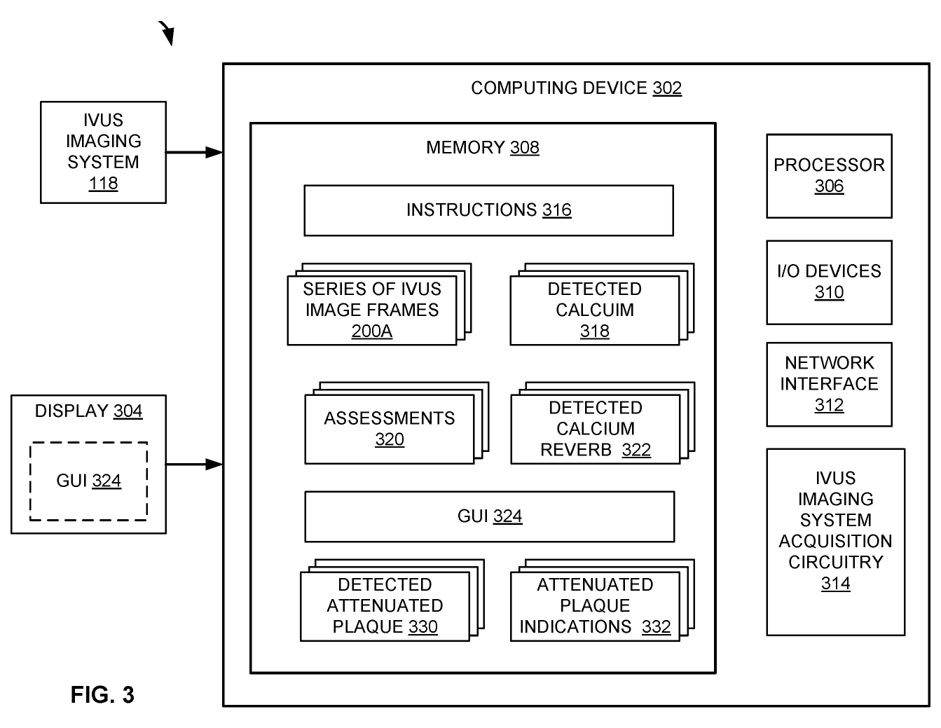

FIG. 3 illustrates an IVUS image visualization system 300, which can be implemented to detect calcium, calcium reverb, and attenuated plaque, and display the locations of the detected calcium, calcium reverb, and attenuated plaque as part of a pre-PCI procedure. In general, IVUS image visualization system 300 is a system for processing, annotating, and/or presenting IVUS images. IVUS image visualization system 300 can be implemented in a commercial IVUS guidance or navigation system, such as, for example, the AVVIGO^{TM} Guidance System available from Boston Scientific®. The present disclosure provides advantages over prior or conventional IVUS navigation systems in that the location(s), extent, and arrangement of attenuated plaque are detected, mapped, and displayed in various graphical form(s), thereby allowing a user (e.g., physician, or the like) to select relevant locations and treatment options and make clinical decisions to improve patient outcomes in a post-PCI procedure.

IVUS image visualization system 300 includes a computing device 302 and display 304. Optionally, IVUS image visualization system 300 includes an IVUS imaging system, such as IVUS imaging system 118. Where IVUS image visualization system 300 includes IVUS imaging system 118, IVUS image visualization system 300 could be implemented as part of control system 122 or alternatively, control system 122 could be implemented as part of computing device 302 of IVUS image visualization system 300. IVUS image visualization system 300 will be described with reference to cardiac vasculature structure 100 and IVUS imaging system 118 of FIGS. 1A and 1B as well as the series of IVUS image frames 200A and longitudinal view 200B of FIGS. 2A and 2B, for clarity and ease of discussion. However, it is noted that IVUS image visualization system 300 could be implemented for use with another IVUS imaging system or even another intravascular imaging system utilizing a different imaging modality than IVUS imaging system 118.

Computing device 302 can be any of a variety of computing devices. In some embodiments, as noted above, computing device 302 can be incorporated into and/or implemented by a console to be coupled to an intravascular imaging device (e.g., IVUS imaging system 118, IVUS imaging catheter 106, or the like). With some embodiments, computing device 302 can be a workstation or server communicatively coupled to IVUS imaging system 118. With still other embodiments, computing device 302 can be provided by a cloud-based computing device, such as, by a computing device as a service system accessible over a network (e.g., the Internet, an intranet, a wide area network, or the like). Computing device 302 can include processor 306, memory 308, input and/or output (I/O) devices 310, network interface 312, and IVUS imaging system acquisition circuitry 314.

Display 304 may include any of a variety of devices arranged to display graphical information, such as, a light emitting diode (LED) display, or the like. It is to be appreciated that although display 304 is depicted separate from computing device 302, display 304 could be implemented as part of computing device 302 or be distinct from computing device 302.

The processor 306 may include circuity or processor logic, such as, for example, any of a variety of commercial processors. In some examples, processor 306 may include multiple processors, a multi-threaded processor, a multi-core processor (whether the multiple cores coexist on the same or separate dies), and/or a multi-processor architecture of some other variety by which multiple physically separate processors are in some way linked. Additionally, in some examples, the processor 306 may include graphics processing portions and may include dedicated memory, multiple-threaded processing and/or some other parallel processing capability. In some examples, the processor 306 may be an application specific integrated circuit (ASIC) or a field programmable integrated circuit (FPGA).

The memory 308 may include logic, a portion of which includes arrays of integrated circuits, forming non-volatile memory to persistently store data or a combination of non-volatile memory and volatile memory. It is to be appreciated, that the memory 308 may be based on any of a variety of technologies. In particular, the arrays of integrated circuits included in memory 308 may be arranged to form one or more types of memory, such as, for example, dynamic random-access memory (DRAM), NAND memory, NOR memory, or the like.

I/O devices 310 can be any of a variety of devices to receive input and/or provide output. For example, I/O devices 310 can include, a keyboard, a mouse, a joystick, a foot pedal, a display, a touch enabled display, a haptic feedback device, an LED, or the like.

Network interface 312 can include logic and/or features to support a communication interface. For example, network interface 312 may include one or more interfaces that operate according to various communication protocols or standards to communicate over direct or network communication links. Direct communications may occur via use of communication protocols or standards described in one or more industry standards (including progenies and variants). For example, network interface 312 may facilitate communication over a bus, such as, for example, peripheral component interconnect express (PCIe), non-volatile memory express (NVMe), universal serial bus (USB), system management bus (SMBus), SAS (e.g., serial attached small computer system interface (SCSI)) interfaces, serial AT attachment (SATA) interfaces, or the like. Additionally, network interface 312 can include logic and/or features to enable communication over a variety of wired or wireless network standards. For example, network interface 312 may be arranged to support wired communication protocols or standards, such as, Ethernet, or the like. As another example, network interface 312 may be arranged to support wireless communication protocols or standards, such as, for example, Wi-Fi, Bluetooth, 5G, or the like.

The IVUS imaging system acquisition circuitry 314 may include circuity including custom manufactured or specially programmed circuitry configured to receive or receive and send signals with IVUS imaging system 118, including indications of intravascular images, intravascular image frames, or a series of intravascular image frames. For example, IVUS imaging system acquisition circuitry 314 can include image acquisition circuitry (not shown) and/or pulse generator 126. Memory 308 can include instructions 316, series of IVUS image frames 200A, detected calcium 318, assessments 320, detected calcium reverb 322, detected attenuated plaque 330, attenuated plaque indications 332, as well as GUI 324.

During operation, processor 306 can execute instructions 316 to cause computing device 302 to receive (e.g., from IVUS imaging system 118, or the like) a recording of an “IVUS run” and store the recording as the series of IVUS image frames 200A in memory 308. For example, processor 306 can execute instructions 316 to receive information elements from IVUS imaging system 118 comprising indications of IVUS image frames 202-1, 202-2, 202-3 to 202-N, captured by IVUS imaging catheter 106 while IVUS imaging catheter 106 is pulled through cardiac artery 102 from distal point 112 to proximal point 114 through the obstruction 116. It is to be appreciated that the series of IVUS image frames 200A can be stored in a variety of image formats or even non-image formats or data structures.

The present disclosure provides to process the series of IVUS image frames 200A to detect calcium, calcium reverb, and attenuated plaque from the series of IVUS image frames 200A and display visual indications of the locations of the detected calcium, calcium reverb, and attenuated plaque via various GUIs. In some examples, processor 306 can execute instructions 316 to receive indications of calcium reverb from series of IVUS image frames 200A and store the indications as detected calcium reverb 322. With some examples, processor 306 can execute instructions 316 to detect the calcium reverb represented in the series of IVUS image frames 200A and store indications of the detected calcium reverb as detected calcium reverb 322. For example, processor 306 can execute instructions 316 to identify frames of series of IVUS image frames 200A (e.g., IVUS image frames 202-2, 202-3, etc.) where calcium is detected and identify areas of IVUS reverberation indicative of calcium thickness in the calcium and store indications of the calcium reverb and/or the frames of the series of IVUS image frames 200A associated with the detected calcium reverb as detected calcium reverb 322. In various embodiments, processor 306 can execute instructions 316 to detect the presence, extent, location(s) and arrangement of attenuated plaque based on the frames of the series of IVUS images, in some embodiments based at least in part on the detection of the presence and locations of calcium and/or calcium reverb.

Details of attenuated plaque detection techniques, such as techniques to identify frames of the series of IVUS image frames 200A in which attenuated plaque can be identified are further described below. In some embodiments, processor 306 can execute instructions 316 to identify the detected calcium 318 and identify detected calcium reverb 322 from the series of IVUS image frames 200A using image processing tool(s) to identify frames of a series of frames in which calcium is represented. As another example, processor 306 can execute instructions 316 to identify the detected calcium reverb 322 from the series of IVUS image frames 200A using image processing algorithms (e.g., image segmentation, etc.). With yet another example, processor 306 can execute instructions 316 to identify the detected calcium reverb 322 from the series of IVUS image frames 200A using a combination of image processing techniques and ML models. For example, processor 306 can execute instructions 316 to apply a cross-sectional segmentation to the frames of the series of IVUS image frames 200A and then infer frames representing calcium reverb from the frame segmentations using an image processing tool. In various embodiments, processor 306 can execute instructions 316 to apply an image processing tool applying vector analysis to detect and identify the location(s), extent, and arrangement of attenuated plaque based on the series of IVUS image frames 200A and may further utilize image processing algorithms to generate a mapping of the location(s), extent, and arrangement of the detected attenuated plaque. The mapping of the attenuated plaque may be used to generate and render for display a graphical depiction of the location(s), extent, arrangement of the detected attenuated plaque.

Further, processor 306 can execute instructions 316 to derive the detected attenuated plaque 330 from the assessments 320. Said differently, processor 306 can execute instructions 316 to derive assessments for each frame of the series of IVUS image frames in which attenuated plaque 330 is detected. In general, the assessments 320 can include vessel and/or lumen borders, raw vessel and/or lumen areas, smoothed vessel and/or lumen areas, a plaque burden, calcium arc length, presence of calcium reverb, and graphical indication(s) illustrative of the location(s) extent, and arrangement of attenuated plaque, or the like. With some embodiments, processor 306 can execute instructions 316 to determine the assessments 320 using image processing techniques, ML models, or a combination of image processing techniques and ML models.

Examples of processing IVUS images to detect calcium may be described in more detail in United States Patent Application Publication No. 2023/0157672, titled “Intravascular Ultrasound Imaging and Calcium Detection Methods” and filed on Mar. 1, 2022; United States Patent Application Publication No. 2023/0112017, titled “Medical Device Systems for Automatic Lesion Assessment” and filed on Oct. 5, 2022; United States Patent Application Publication No. 2023/0380806, titled “Systems and Methods for Intravascular Visualization” and filed on May. 26, 2023; United States Patent Application Publication No. 2024/0081781, which applications are each incorporated herein by reference in their entirety.

Processor 306 can execute instructions 316 to generate attenuated plaque indication 332 for detected attenuated plaque 330, and to generate GUI 324 from the attenuated plaque indicators 332. In general, the attenuated plaque indications 332 and/or GUI 324 can comprise visual indications of the series of IVUS image frames 200A, the detected attenuated plaque indicators 332, and the assessments 320. The areas with detected attenuated plaque 330 and/or assessments 320 can be visually depicted in GUI 324.

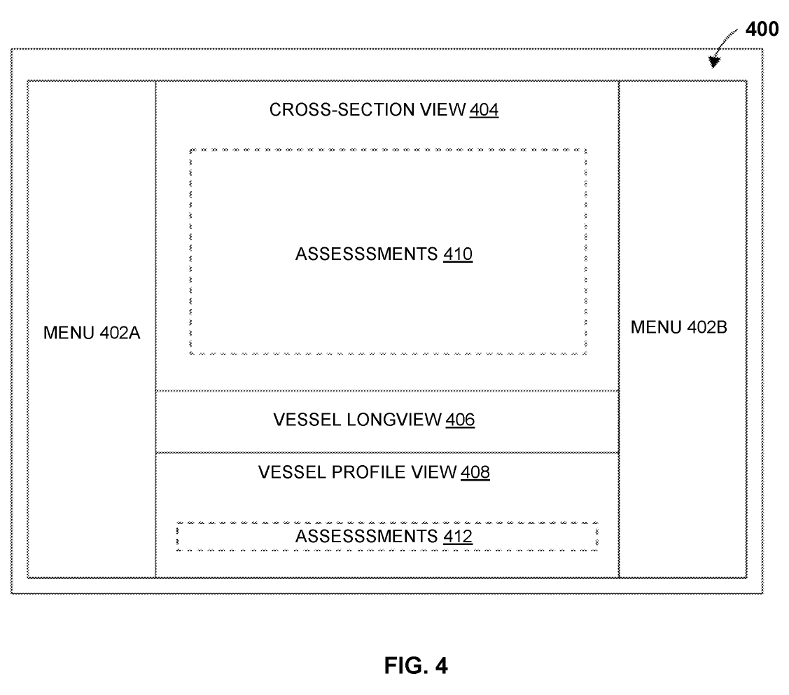

FIG. 4 illustrates a first GUI 400, which can be generated according to some embodiments of the present disclosure. In some embodiments, GUI 400 can be generated by IVUS image visualization system 300 as GUI 324 and displayed on display 304. As depicted, GUI 400 includes several graphical components or visual indications, such as, menus 402A and 402B, cross-section view 404, and vessel long view 406, and vessel profile view 408.

With some embodiments, a processor (e.g., processor 124, processor 306, or the like) can be configured to execute instructions (e.g., instructions 316, or the like) to generate GUI 400. In some examples, GUI 400 can be generated responsive to an automatic attenuated plaque detection process. For example, with some embodiments, IVUS image visualization system 300 can be arranged to automatically detect attenuated plaque in cardiac artery 102 from the series of IVUS image frames 200A and identify locations of attenuated plaque (e.g., via machine learning, image classification, artificial intelligence, or the like). Responsive to this detection, GUI 400 could be generated to include visual indications of the location(s), extent, and arrangement of the detected attenuated plaque.

Menu 402A can comprise GUI inputs such as buttons, drop down menus, selection icons, or the like. Menu 402A can include GUI input options to select measurement and annotation tools, length tools, modification reset buttons, or the like. In various embodiments, Menu 402A can further include a Bookmark input operable, responsive to user actuation, to save a current longitudinal position along a longitudinal axis of the longitudinal view as a bookmark, the bookmark being associated with the saved longitudinal position and being subsequently recallable to drive a cross-sectional view of the series of IVUS image frames 200A corresponding to the saved longitudinal position. Menu 402B can comprise GUI inputs such as buttons, drop down menus, selection icons, or the like. Menu 402B can include GUI inputs options to select views related to views of the IVUS images, layout options, annotations, navigation, dynamic review options, status of the computing device, or the like.

Cross-section view 404 can comprise a cross-sectional view, or on-axis view, of one (e.g., a frame, or the like) of the series of IVUS image frames 200A. For example, cross-section view 404 can include IVUS image frame 202-1 (or IVUS image frame 202-2 to 202-N). Further, cross-section view 404 can include assessments 410. With some examples, the assessments can include visual indications of vessel and/or lumen borders, indications of vessel and/or lumen area, indications of a plaque burden, indications of a calcium arc length, and/or a calcium reverb indication, and/or attenuated plaque indication 332.

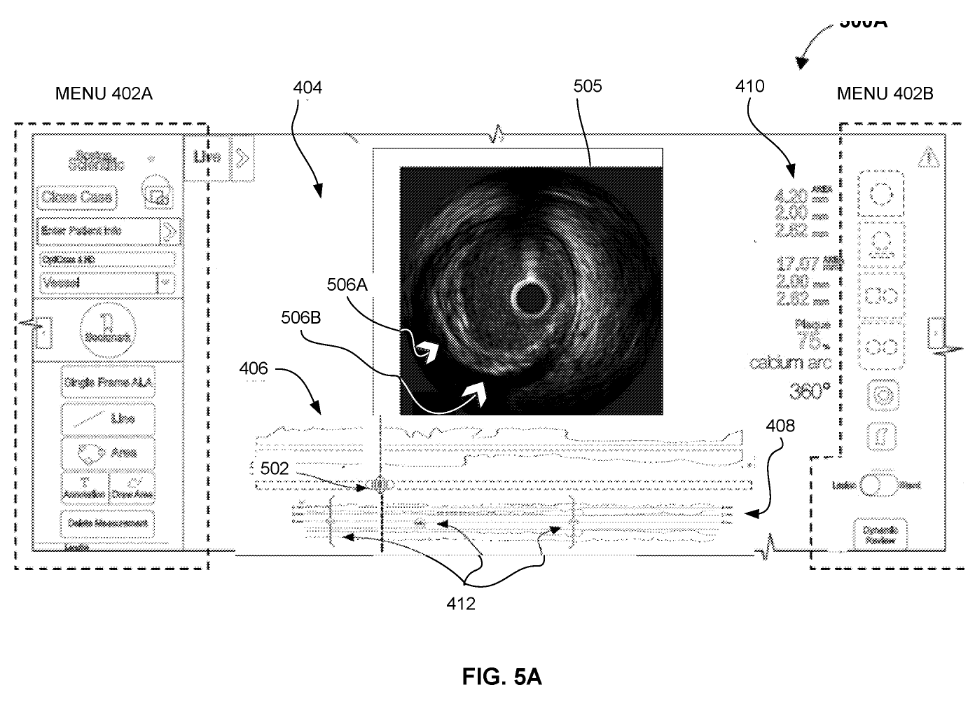

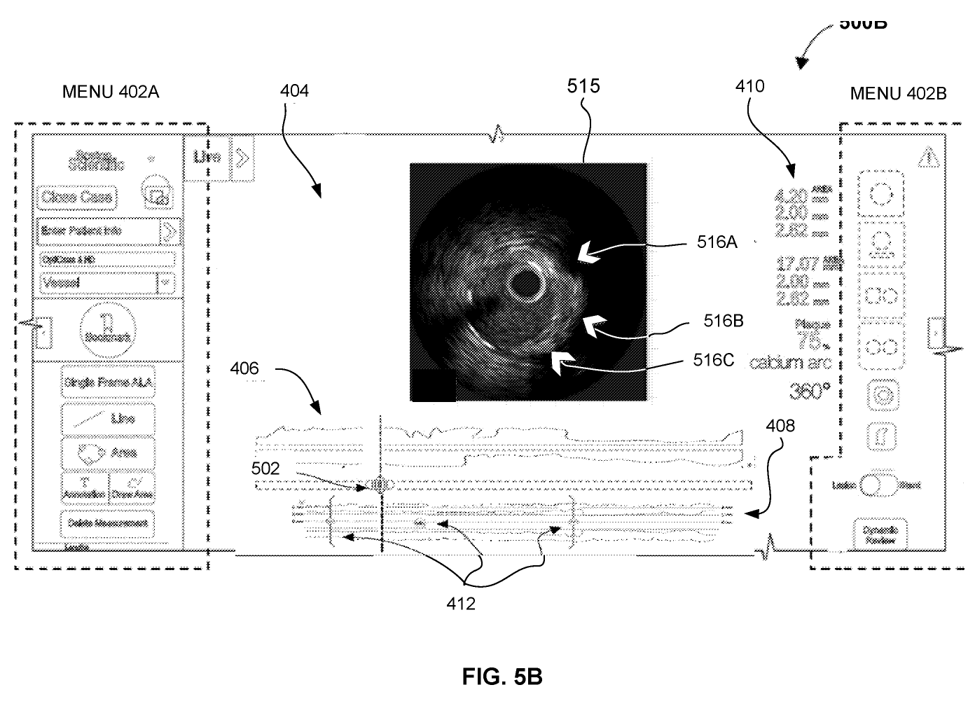

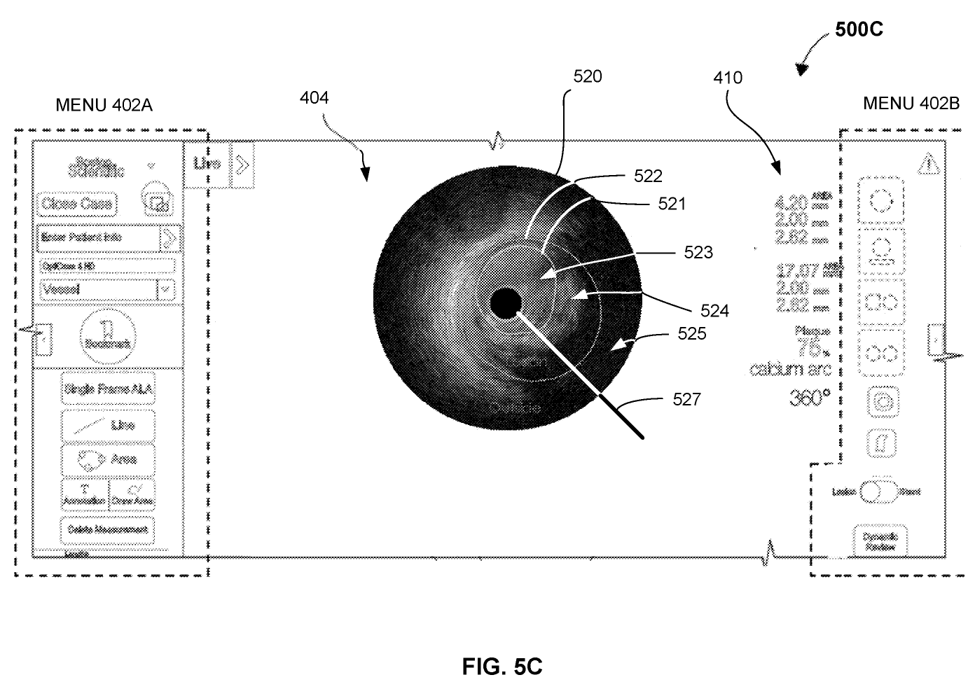

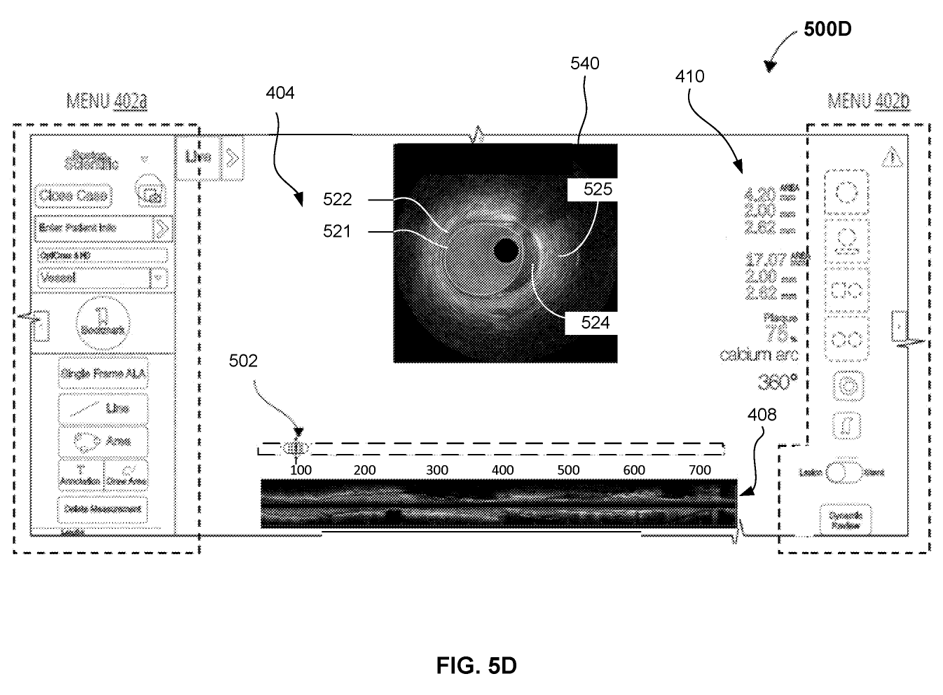

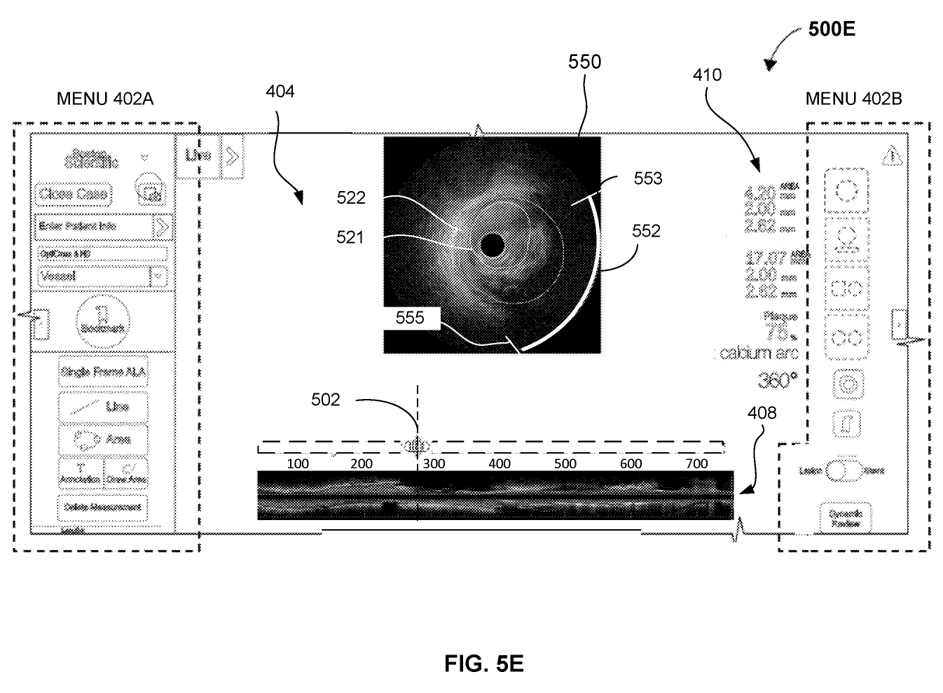

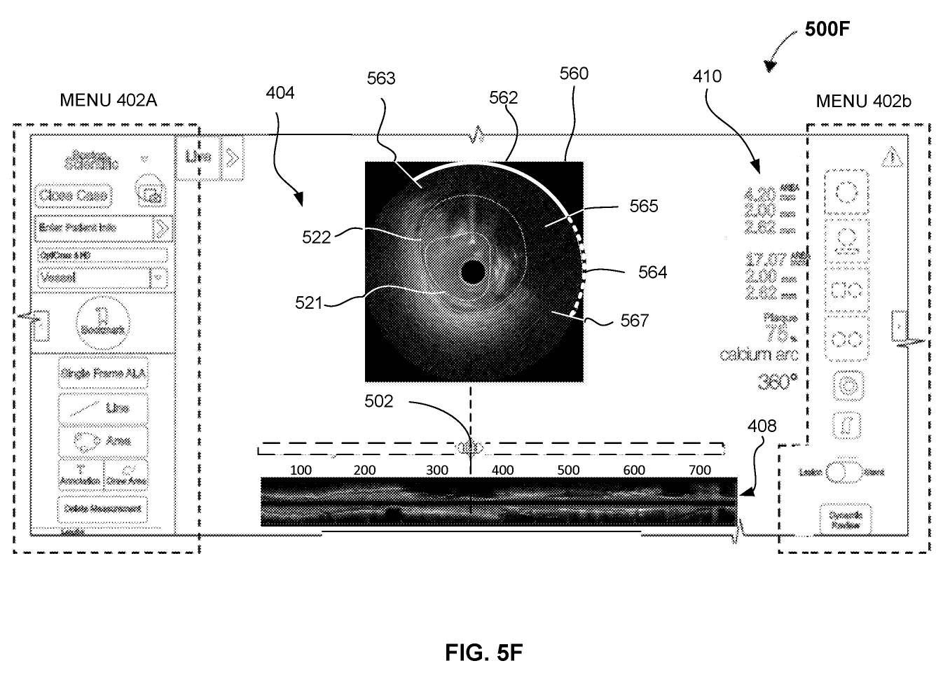

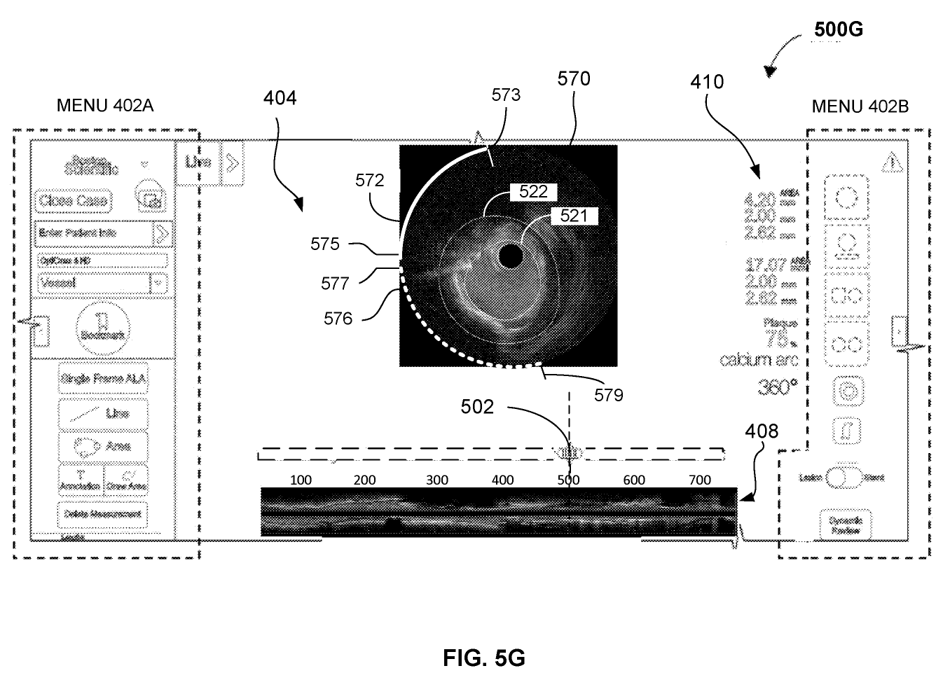

Vessel long view 406 can include the series of IVUS image frames 200A represented longitudinally, for example, as depicted in longitudinal view 200B of FIG. 2B. Vessel profile view 408 can include a visual depiction of the longitudinal profile of the vessel. For example, vessel profile view 408 can include indications of the lumen border and vessel border, indications of plaque, indications of a stent, indications of key frames, or the like. Further, vessel long view 406 and vessel profile view 408 can include a navigation slider (see e.g., FIGS. 5A-5G), which can be selected and used by a user (e.g., physician, or the like) to navigate through the series of IVUS image frames 200A. In some examples, the slider can be linked to the cross-section view 404 such that the frame depicted in cross-section view 404 corresponds to the location of the slider along longitudinal view 406. That is, as the slider is moved the image displayed in cross-section view 404 can change to match the location of the slider.

Further, vessel long view 406 and vessel profile view 408 can include assessments 412. With some examples, assessments 412 can include visual indications of plaque, plaque burden, calcium, detected calcium reverb, detected attenuated plaque, stent expansion, or the like.

FIG. 5A illustrates a second example GUI 500A, which can be generated according to some embodiments of the present disclosure. For example, GUI 500A can be generated by IVUS image visualization system 300 as GUI 324 and displayed on display 304 as image 505 of a frame image of a vessel in cross-section taken using IVUS imaging process. With some embodiments, responsive to receiving the series of IVUS image frames 200A, processor 306 can execute instructions 316 to generate image 505.

GUI 500A can include menus 402A and 402B, cross-section view 404, vessel longview 406, and vessel profile view 408. In vessel long view 406, slider 502 is represented. GUI 500A provides slider 502 such that a user can scrub across the series of IVUS image frames 200A. It is to be appreciated that slider 502 is one example of a user-positionable longitudinal marker positionable along a longitudinal axis of vessel long view 406, and in various embodiments the user-positionable longitudinal marker may alternatively or additionally comprise a bookmark associated with a saved longitudinal position along the longitudinal axis, a draggable pin marker positionable along the longitudinal axis, or another graphical element configured to be positioned along the longitudinal axis. Further, in cross-section view 404 a frame of the series of IVUS image frames 200A (e.g., corresponding to the slider location, or the like) is depicted along with various assessments 410 (e.g., lumen and vessel borders, area, plaque burden, calcium arc, etc.). Additionally, GUI 500A can include assessments 412 in vessel long view 406. With some examples, assessments 412 can include visual indications of key frames (e.g., brackets, or the like), a visual indication of the location of the minimum lumen area, etc.

As shown in FIG. 5A, areas indicated by chevrons 506A and 506B include areas of attenuated plaque. Because of the dark nature of the image in these areas, as well as a lack of contrast in the image to other features depicted in the image, it can be difficult to determine, for example using only manual inspection of the image, where these areas of attenuated plaque exist, and the extent and boundaries of the attenuated plaque. Embodiments as described herein include systems, methods, and techniques for detecting the location, extent, arrangement, and boundaries of attenuated plaque based on further analysis of the IVUS generated images, such as image 505, and to provide graphical information about these detected parameters associated with the attenuated plaque, which may then be displayed as part of a GUI, as further described below.

FIG. 5B illustrates a third example GUI 500B, which can be generated according to some embodiments of the present disclosure. For example, GUI 500B can be generated by IVUS image visualization system 300 as GUI 324 and displayed on display 304. With some embodiments, responsive to receiving the series of IVUS image frames 200A, processor 306 can execute instructions 316 to generate image 515.

Like GUI 500A, GUI 500B can include menus 402A and 402B, cross-section view 404, vessel long view 406, and vessel profile view 408. GUI 500B likewise includes slider 502, a frame of the series of IVUS image frames 200A depicted in the cross-section view 404 and the vessel profile view 408 depicted below the vessel long view 406. Likewise, GUI 500B includes assessments 410, assessments 412.

As shown in FIG. 5B, areas indicated by chevrons 516A, 516B, and 516C include areas of attenuated plaque. Again, because of the dark nature of the image in these areas, as well as a lack of contrast in the image to other features depicted in the image, it can be difficult to determine, for example using only manual inspection of the image, where these areas of attenuated plaque exist, and the extent and boundaries of the attenuated plaque. Embodiments as described herein include systems, methods, and techniques for detecting the location, extent, arrangement, and boundaries of attenuated plaque based on further analysis of the IVUS generated images, such as image 515, and to provide graphical information about these detected parameters associated with the attenuated plaque, which may then be displayed as part of a GUI, as further described below.

FIG. 5C illustrates a fourth example GUI 500C, which can be generated according to some embodiments of the present disclosure. For example, GUI 500C can be generated by IVUS image visualization system 300 as GUI 324 and displayed on display 304. With some embodiments, responsive to receiving the series of IVUS image frames 200A, processor 306 can execute instructions 316 to detect attenuated plaque. and generate detected attenuated plaque 330 (FIG. 3). Further, processor 306 can execute instructions 316 to a plurality of GUIs, as illustrated and described below with respect to FIGS. 5D-5G, comprising visual indications of the detected attenuated plaque 330. While embodiments of GUI of GUI 500C may include any of the elements, such as menus 402A, 402B, vessel long view 408, vessel profile view 408, and slider 502, some of these elements are not shown in FIG. 5C for clarity purposes.

As shown in FIG. 5C, cross-sectional view area 404 includes a cross-sectional image 520. Image 520 can be any cross-sectional image along the vessel profile and generated as an image frame provided by an IVUS imaging process. An analysis process of image 520 utilized to detect and map the location of attenuated plaque located in a vessel of a patient at the longitudinal position of the vessel represented in image 520 will be further described below.

Analysis of image 520 may begin with determining a first border 521 and a second border 522. Border 521 represents the location of the wall of the lumen extending through the vessel being represented in image 520, which forms a closed ring-shaped border. The ring-shaped border 521 encloses an internal area 523, representing the open area of the vessel formed by the lumen. The second border 522 represents the location of the wall of the vessel at the portion of the vessel being represented in image 520. Second border 522 forms an enclosed ring-shaped border, which extends around the area outside first border 521, and completely encircles the first border 521. An intra-border area 524 of the image includes portions of the image that are outside of the first border 521 but are also inside second border 523. In other words, the potions of the image that are between first border 521 and second border 522 are included in the intra-border area 524. A third exterior area 525 includes all of the areas of image 520 that lie outside of and are not enclosed within second border 522.

Once first border 521 and second border 522 have been defined for the particular cross-sectional image 520, an analysis, for example using an image processing application, may be performed to determine the presence and location of calcium within the intra-border area 524. Any method may be used to detect the presence and location of calcium, and any detected calcium is mapped into the location(s) and arrangements of the detected calcium within image 520. Next, any calcium reverberations are detected and may be filtered out of the image using any available filtering techniques. In various embodiments, any calcium that has been detected and mapped, and any calcium reverb has been filtered out of the image, an analysis is performed to determine if any attenuated plaque is indicated based on the information provided by the now processed image 520. In various embodiments, as a first step in this analysis, areas that have been determined to include calcium are eliminated as possible locations for attenuated plaque. Next, areas within the image are analyzed to determine areas where no echo response was received back as a result of impinging a signal, such as an ultrasound signal generated by a transducer positioned within the portion of the vessel associated with image 520, such as transducer 110 (FIG. 1). However, in alternative embodiments, the analysis of the images may include image processing to simultaneously determine the presence of calcium, to detect the presence of calcium reverberation, and/or detect the presence and location(s) of attenuated plaque.

The lack of echo returns may be determined based on comparing areas of the image within the intra-border area 524 to determine if a minimum level of intensity for a response was recorded for each of the areas where no calcium was detected and from which no echo or little echo response was received. For these areas where little or no echo was received back, these areas potentially represent areas where attenuated plaque is located in the vessel. Due to the property of attenuated plaque attenuating the ultrasound signal originating from the ultrasound transmitter, any echo signal that might be reflected back to the transducer by tissue that is further away from the transducer and having the attenuated plaque positioned between the transducer and the tissue will also prevent the echo signal from reflecting a signal back to the ultrasound receiver. For example, tissue or other fibers located beyond the second border 522, and thus outside of the vessel, would normally provide an echo response as a reflection of the ultrasound signal emitted from the ultrasound transmitter. However, the signal, being attenuated by first passing through the attenuated plaque layer, is not reflected back to the receiver at an expected level of intensity.

Using this principle, for any areas within the intra-border area 524 of the image that are dark due to lack a minimum level of response, an analysis is done to determine a level or levels of intensity for areas aligned along a same vector extending from the point of transmission of the ultrasound signal and the area where no echo was received. If these corresponding areas in outside area 525 also do not indicate some minimum level of echo response, the area within intra-border area 524 is determined to include attenuated plaque. By performing this analysis at various vector angles around the image 520, the location of the attenuated plaque, such as the rotational positioning, a start position, and stop position of the attenuated plaque, for example an arc length of the attenuated plaque, may be mapped relative to image 520. In addition, by further analysis of the areas within intra-border area 524, a thickness for the attenuated plaque at each rotational position within image 520 may be determined. By way of illustration, a vector 527 may be constructed extending from the point in image 520 where the ultrasound transducer was positioned within lumen of the vessel outward through an area within intra-border areas 524 where no calcium was detected, and no echo signal was received back at the transducer. An analysis is then performed, in some embodiments using image processing and/or artificial intelligence to determine a level of intensity depicted in the image along the area of vector 527 that extends into outer area 525. In various embodiments, if a threshold level of echo intensity is not provided along vector 527 in the area where vector 527 extends into outer area 525, the corresponding area along vector 527 that extends through the dark portion of the image within intra-border area 524 is determined to comprise attenuated plaque.

In various embodiments, the above-described analysis used to detect and map the presence and location(s) of attenuated plaque may be performed by a software tool, such as a machine learning tool, use of neural network(s), an artificial intelligence application, or some combination of these or other tools, such as image processing tools. Once the analysis of image 520 is completed, a mapping of any detected attenuated plaque may be generated, and graphical indications may be superimposed onto image 520 with information indicative of the location, thickness, and arrangement of the attenuated plaque at the longitudinal location within the vessel being represented by image 520. In various embodiments, the GUI 500C generated and displayed with the graphical information superimposed on the image 520 related to detected and mapped attenuated plaque may also information in assessment 410 displayed as part of the GUI. In various embodiments, assessment information 410 may include information, which may be in the form of text information, which may indicate values for parameters such as arc length(s), rotational position(s), thicknesses, and other information related to the attenuated plaque detected and mapped for the portion of the vessel represented in image 520.

In various embodiments, the analysis to detect and map attenuated plaque can be performed on some image frames, or an entire series of image frames included as images of a length of a vessel of a patient, such as a length of vessel 102 between distal point 112 and proximal point 114, as illustrated and described above with respect to FIG. 1. In various embodiments, a mapping of the detected attenuated plaque along the entirety of the imaged length of the vessel may be generated as graphical component and displayed as part of a GUI.

Additional examples of GUIs that display various characteristic of attenuated plaque, or lack thereof, in IVUS images are further illustrated and described below with respect to FIGS. 5D–5G and 6-8.

FIG. 5D illustrates a fifth example GUI 500D, which can be generated according to some embodiments of the present disclosure. For example, GUI 500D can be generated by IVUS image visualization system 300 as GUI 324 and displayed on display 304. With some embodiments, responsive to receiving the series of IVUS image frames 200A, processor 306 can execute instructions 316 to detect obstruction 116 and generate detected calcium 318. Further, processor 306 can execute instructions 316 to generate GUI 500D comprising visual indications of any detected attenuated plaque 330.

Like GUIs 500A, 500B and 500C, GUI 500D can include menus 402A and 402B, cross-sectional view 404, vessel long view 406, vessel profile view 408, assessments 412, as well as slider 502, although one or more of these features may not be shown in FIG. 5D for clarity purposes. GUI 500D includes a cross-sectional image 540 included in cross-sectional view 404. Cross-sectional image 540 represents image data that was imaged from the vessel depicted in vessel profile 408 when imaging the vessel at the position of slider 502 in GUI 500D. As depicted, this position and the cross-sectional image 540 corresponds to the 100^{th} frame of the series of IVUS image frames 200A. Cross-sectional image 540 includes a graphical rendering of a first border 521 and a second border 522, which defines intra-border area 524 and outer area 525.

In various embodiments, the analysis to detect the presence of attenuated plaque at the longitudinal location within the vessel represented in image 540 can be performed, and the results graphically depicted as part of GUI 500D. In this particular instance, no calcium and no attenuated plaque were detected at the location of the vessel being represented by image 540. In such instances, assessment 410 may provide an indication, for example using displayed text, indicating the no calcium and no attenuated plaque were detected as being present at the portion of the vessel, in this case the 100^{th} frame of the series of IVUS image frames 200A (e.g., based on the analysis performed on image 540).

FIG. 5E illustrates a sixth example GUI 500E, which can be generated according to some embodiments of the present disclosure. For example, GUI 500E can be generated by IVUS image visualization system 300 as GUI 324 and displayed on display 304. With some embodiments, responsive to receiving the series of IVUS image frames 200A, processor 306 can execute instructions 316 to detect obstruction 116 and generate detected calcium 318. Further, processor 306 can execute instructions 316 to generate GUI 500E comprising visual indications of any detected attenuated plaque 330.

Like GUIs 500A, 500B, 500C, and 500D, GUI 500E can include menus 402A and 402B, cross-sectional view 404, vessel long view 406, vessel profile view 408, assessments 410, as well as slider 502, although one or more of these features may not be shown in FIG. 5E for clarity purposes. GUI 500E includes a cross-sectional image 550 included in cross-sectional view 404. Cross-sectional image 550 represents image data that was imaged from the vessel depicted in vessel profile 408 when imaging the vessel at the position of slider 502 in GUI 500E. As depicted, this position and the cross-sectional image 550 corresponds to the 275^{th} frame of the series of IVUS image frames 200A.

Cross-sectional image 550 includes a graphical rendering of first border 521 and a second border 522, which defines intra-border area 524 and outside area 525.

In various embodiments, the analysis to detect the presence of attenuated plaque at the longitudinal location within the vessel represented in image 550 can be performed, and the results graphically depicted as part of GUI 500E. In this particular instance, attenuated plaque was detected at the longitudinal location of the vessel being represented by image 550. In such instances, GUI 500E may include graphical information generated and displayed as part of the GUI representative of one or more aspects of the detected attenuated plaque. In one non-limiting example, a solid line arc 552, extending between end points 553 and 555, may be rendered onto image 550 to represent the rotational positioning of the attenuated plaque within the potion of the vessel being represented in image 550. In various embodiments, end point 553 and 555 represent the points where the detected attenuated plaque begins and ends, respectively, relative to the circumference around the vessel.

In addition to the graphical information that may be superimposed onto image 550 as described above, assessment 410 in GUI 500E may include additional information, such as text and/or other type of graphical information, related to the detected attenuated plaque. For example, assessment 410 may include an indication that no calcium was detected, and that attenuated plaque was detected. Assessment 410 may include additional information related to the detected attenuated plaque, such as arc length of the attenuated plaque between end point 553 and 555, thickness values for the attenuated plaque, such as a maximum thickness value, and positional information, such as the rotational positioning of the attenuated plaque around the circumference of the vessel in the cross-sectional are of the vessel represented by image 550. The types and formats of information provided in assessment 410 is not limited to any particular type of information or to a particular way of formatting of the information being provided and may include any information and format for the information deemed desirable for the conveyance of information related opt the results of the analysis of image 550.

FIG. 5F illustrates a seventh example GUI 500F, which can be generated according to some embodiments of the present disclosure. For example, GUI 500F can be generated by IVUS image visualization system 300 as GUI 324 and displayed on display 304. With some embodiments, responsive to receiving the series of IVUS image frames 200A, processor 306 can execute instructions 316 to detect obstruction 116 and generate detected calcium 318. Further, processor 306 can execute instructions 316 to generate GUI 500F comprising visual indications of any detected attenuated plaque 330.

Like GUIs 500A, 500B 500C, 500D, and 500E, GUI 500F can include menus 402A and 402B, cross-sectional view 404, vessel long view 406, vessel profile view 408, assessments 410, as well as slider 502, although one or more of these features may not be shown in FIG. 5F for clarity purposes. GUI 500F includes a cross-sectional image 560 included in cross-sectional view 404. Cross-sectional image 560 represents image data that was imaged from the vessel depicted in vessel profile 408 when imaging the vessel at the position of slider 502 in GUI 500F. As depicted, this position and the cross-sectional image 560 corresponds to the 350^{th} frame of the series of IVUS image frames 200A. Further, as depicted, cross-sectional image 560 includes a graphical rendering of a first border 521 and a second border 522, which defines intra-border area 524 and outside area 525.

In various embodiments, the analysis to detect the presence of attenuated plaque at the longitudinal location within the vessel represented in image 560 can be performed, and the results graphically depicted as part of GUI 500F. In this particular instance, both calcium and attenuated plaque were detected at the longitudinal location of the vessel being represented by image 560. In such instances, GUI 500F may include graphical information generated and displayed as part of the GUI representative of one or more aspects of the detected calcium and one or more aspects of the detected attenuated plaque. In one non-limiting example, a solid line arc 562, extending between end points 563 and 565, may be rendered onto image 560 to represent the rotational positioning of the attenuated plaque within the potion of the vessel being represented in image 550. In various embodiments, end point 563 and 565 represent the points where the detected attenuated plaque begins and ends, respectively, relative to the circumference around the vessel. In addition, as shown in GUI 500F a dashed line arc 564, extending between end points 565 and 567, may be rendered onto image 560 to represent the rotational positioning of the detected calcium within the potion of the vessel being represented in image 560. In various embodiments, end point 565 and 567 represent the vector points where the detected calcium begins and ends, respectively, relative to the circumference around the vessel.

In addition to the graphical information that may be superimposed onto image 560 as described above, assessment 410 in GUI 500F may include additional information, such as text and/or other type of graphical information, related to the detected attenuated plaque. For example, assessment 410 may include an indication that calcium was detected, and that attenuated plaque was also detected at the location within the vessel represented by image 560. Assessment 410 may include additional information related to the detected calcium and/or the detected attenuated plaque, such as arc lengths of the detected calcium and the attenuated plaque, thickness values for the calcium and for the attenuated plaque, such as a maximum thickness values for each, and positional information, such as the rotational positioning of the calcium and for the attenuated plaque around the circumference of the vessel in the cross-sectional are of the vessel represented by image 560. The types and formats of information provided in assessment 410 is not limited to any particular type of information or to a particular way of formatting of the information being provided and may include any information and format for the information deemed desirable for the conveyance of information related opt the results of the analysis of image 560.

FIG. 5G illustrates an eighth example GUI 500G, which can be generated according to some embodiments of the present disclosure. For example, GUI 500G can be generated by IVUS image visualization system 300 as GUI 324 and displayed on display 304. With some embodiments, responsive to receiving the series of IVUS image frames 200A, processor 306 can execute instructions 316 to detect obstruction 116 and generate detected calcium 318. Further, processor 306 can execute instructions 316 to generate GUI 500G comprising visual indications of any detected attenuated plaque 330.

Like GUIs 500A, 500B 500C, 500D, 500E, and 500F, GUI 500G can include menus 402A and 402B, cross-sectional view 404, vessel long view 406, vessel profile view 408, assessments 410, as well as slider 502, although one or more of these features may not be shown in FIG. 5G for clarity purposes. GUI 500G includes a cross-sectional image 570 included in cross-sectional view 404. Cross-sectional image 570 represents image data that was imaged from the vessel depicted in vessel profile 408 when imaging the vessel at the position of slider 502 in GUI 500G. As depicted, this position and the cross-sectional image 570 corresponds to the 500^{th} frame of the series of IVUS image frames 200A. Further, as depicted, cross-sectional image 570 includes a graphical rendering of a first border 521 and a second border 522, which defines intra-border area 524 and outside area 525.

In various embodiments, the analysis to detect the presence of attenuated plaque at the location within the vessel represented in image 570 can be performed, and the results graphically depicted as part of GUI 500G. In a result similar to that depicted in GUI 500F, wherein in the instance of GUI 500G, both calcium and attenuated plaque were detected at the longitudinal location of the vessel being represented by image 570. In such instances, GUI 500G may include graphical information generated and displayed as part of the GUI representative of one or more aspects of the detected calcium and one or more aspects of the detected attenuated plaque. In one non-limiting example, a solid line arc 572, extending between end points 573 and 575, may be rendered onto image 570 to represent the rotational positioning of the attenuated plaque within the potion of the vessel being represented in image 570. In various embodiments, end point 573 and 575 represent the points where the detected attenuated plaque begins and ends, respectively, relative to the circumference around the vessel. In addition, as shown in GUI 500G a dashed line arc 576, extending between end points 577 and 579, may be rendered onto image 570 to represent the rotational positioning of the detected calcium within the potion of the vessel being represented in image 570. In various embodiments, end point 575 and 579 represent the points where the detected calcium begins and ends, respectively, relative to the circumference around the vessel.

In addition to the graphical information that may be superimposed onto image 570 as described above, assessment 410 in GUI 500G may include additional information, such as text and/or other type of graphical information, related to the detected attenuated plaque, in as same or similar manner as described above with respect to assessment 410 in GUI 500F. For example, assessment 410 may include an indication that calcium was detected, and that attenuated plaque was also detected. Assessment 410 may include additional information related to the detected calcium and/or the detected attenuated plaque, such as arc lengths of the detected calcium and the attenuated plaque, thickness values for the calcium and for the attenuated plaque, such as a maximum thickness values for each, and positional information, such as the rotational positioning of the calcium and for the attenuated plaque around the circumference of the vessel in the cross-sectional are of the vessel represented by image 570. The types and formats of information provided in assessment 410 is not limited to any particular type of information or to a particular way of formatting of the information being provided and may include any information and format for the information deemed desirable for the conveyance of information related opt the results of the analysis of image 570.

For any of the GUIs described above, the information that may be generated and displayed related to detection of attenuated plaque based on the performing analysis of the IVUS image frames may utilize any of the methods, techniques, and tool described above, or a combination thereof, to perform the detection of attenuated plaque based on the information provided by the image frames.

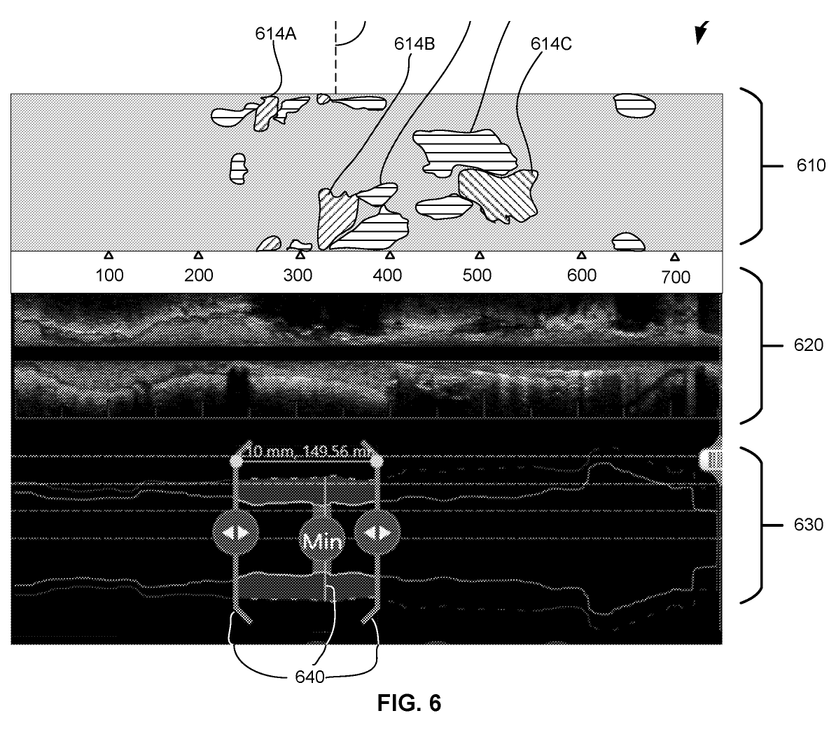

FIG. 6 illustrates a ninth example GUI 600, which can be generated according to some embodiments of the present disclosure. For example, GUI 600 can be generated by IVUS image visualization system 300 as GUI 324 and displayed on display 304. With some embodiments, responsive to receiving the series of IVUS image frames 200A, processor 306 can execute instructions 316 to generate a profile mapping 630 corresponding to a vessel longview 620 generated using a series of intravascular ultrasound (IVUS) images of a vessel of a patient, the series of images analyzed using the methods and techniques described above, to generate a mapping 610 of the locations of both the calcium and any attenuated plaque along the longitudinal length of the imaged portion of the vessel.

As shown in FIG. 6, the vessel longview 620 includes a compilation of the IVUS images taken at least between the 100 and the 700 marks shown in FIG. 6. Mapping 610 represent an image of the circumference of the vessel at any vertical position along the mapping, which is split along a longitudinal axis of the vessel and laid open in a flat layer. Portions of the vessel as mapped in mapping 610 that include larger amounts of calcium are graphically depicted using slanted lines and are illustratively represented by areas 612A and 612B in the mapping. Portions of the vessel as mapped in mapping 610 that include larger amounts of attenuated plaque are graphically depicted using a crosshatch pattern, and are illustratively represented in by areas 614A, 614B, and 614C in the mapping. In various embodiments, mapping 610 and vessel long view 620 are provided as part of a GUI, such as but not limited to GUIs 500A–500G, which include an image of the vessel in cross-section provided in the cross-sectional view 404 portion of the GUI. When provided in conjunction with a GUI that also includes a cross-sectional view, the assessment 640 may be actuated in a same or similar manner as described above with respect to assessment 412, to manipulate which portion of the vessel as mapped in mapping 610 will appear in cross-sectional image.

For example, assessment 640 may be configured to be actuatable so that the view in cross-section corresponds to the portion of mapping 610 indicted by dashed line 642 and therefore includes a graphical depiction of the attenuated plaque 614B at the particular portion of the vessel graphically illustrated in cross-section. This feature allows a user, such as a medical professional, to quickly review mapping 610 and understand the status of the vessel with respect to the presence, extent, and location(s) of areas within the vessel where attenuated plaque has been detected.

In addition, the mapping 610 itself provides an easy to understand and overall picture of the status of the portion of the vessel depicted in the longview 620 and as graphically represented by mapping 610. This comprehensive graphical tool can allow medical professionals, such as a cardiologist, to quickly access information that would suggest what the current condition of the patient may be, what actions and types of procedures, if any, would be most appropriate for the patient, and to direct the procedure to the area(s) within the vessel where the treatment and/or procedure can provide the most effective efficacy.

The ability to generate and display detection of the presence, extent, and location(s) of attenuated plaque within an imaged portion of a vessel, as described above with respect to mapping 610, provides an improvement to the technical field of IVUS imaging, and the display of the mapping of the detected attenuated plaque provides a practical application of the data processing performed in the process of detecting the location(s) and arrangement of any attenuated plaque in a vessel of a patient.

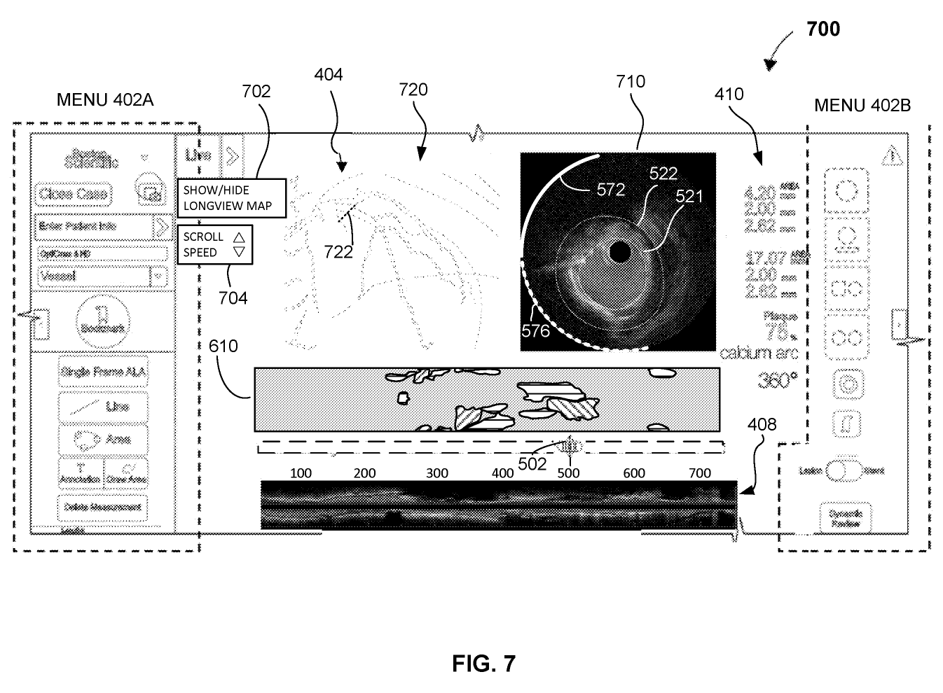

FIG. 7 illustrates a tenth example GUI 700, which can be generated according to some embodiments of the present disclosure. For example, GUI 700 can be generated by IVUS image visualization system 300 as GUI 324 and displayed on display 304. With some embodiments, responsive to receiving the series of IVUS image frames 200A, processor 306 can execute instructions 316 to detect obstruction 116 and generate detected calcium 318. Further, processor 306 can execute instructions 316 to generate GUI 700 comprising visual indications of the detected attenuated plaque 330. With some embodiments, responsive to receiving the series of IVUS image frames 200A, processor 306 can execute instructions 316 to generate a profile mapping 610 corresponding to a vessel longview 620 generated using a series of intravascular ultrasound (IVUS) images of a vessel of a patient, the series of images analyzed using the methods and techniques described above, to generate a mapping 610 of the locations of both the calcium and any attenuated plaque along the longitudinal length of the imaged portion of the vessel.

As shown in FIG. 7, GUI 700 includes a combination of various graphical elements. Embodiments of GUI 700 can include menus 402a and 402b, cross-sectional view 404, vessel profile view 408, assessments 410, as well as slider 502. Further, GUI 700 includes a cross-sectional image 710 included in cross-sectional view 404. Cross-sectional image 710 represents image data that was imaged from the vessel depicted in vessel profile 408 when imaging the vessel at the position of slider 502 in GUI 500F. As depicted, this position and the cross-sectional image 710 corresponds to the 500^{th} frame of the series of IVUS image frames 200A. Cross-sectional image 710 includes a graphical rendering of a first border 521 and a second border 522, which defines intra-border area 524 and outside area 525, as well as graphical indications representing the areas within the vessel where both calcium and attenuated plaque were detected. Assessment 410 may include information related to the detected calcium and the detected attenuated plaque, according to any of the embodiments of GUIs described herein.

GUI 700 also illustrates an example of how the mapping of attenuated plaque may also be provided as part of the displayed graphical information. As shown in FIG. 7, mapping 610 is displayed in a horizontal orientation just above the slider bar that includes slider 502. In various embodiments, the slider 502 can be actuated in a left or right direction across GUI 700, and as slider 502 is moved, a corresponding cross-sectional image will be displayed at cross-sectional image 710. In addition, the positioning of slider bar 502 corresponds to the mapping provided in mapping 610. As such a user such as a physician can actuate slider bar 502 to correspond to a location in mapping 610 where attenuated plaque is indicated, and thereby also cause image 710 to display, in cross-section, of the portion of the vessel indicated by the position of the slider bar. The ability to visually see where the attenuated plaque has been detected at all of the locations along a length of the vessel included in the imaged long view 408, and to move the slider 502 to automatically see an image of any position of the slider bar in cross-section, provides a useful and improved tool for determining the status of a vessels imaged using IVUS imaging techniques. In various embodiments, in addition to changing the cross-sectional view 710 in response to movement of slider 502, an image of where the cross-sectional image 710 is positioned in the artery structure itself may be provided as a graphical display 720. In graphical display 720, a graphical indicator, such as dashed line 722, may be generated and displayed to indicate the position within the artery where the image 710 is representative of the condition of the artery at that particular location.

Embodiments of GUI 700 may include a toggle button 702 that allows for control over showing or hiding the mapping 610 as part of the display being provided by GUI 700. In addition to manually actuating slider bar 502, for example using voice commands or input to the computer system generating GUI 700, embodiments of GUI 700 may be configured to automatically scroll the position of slider bar 502, at some predetermined rate, while the image provided as image 710 and the indication of the portion of the vessel being represented by image 710 changes with the automatic movement of the slider bar 502. In various embodiments, a rate of travel of the automated movement of slider bar 502 may be controlled through input provided to scroll speed button 704.

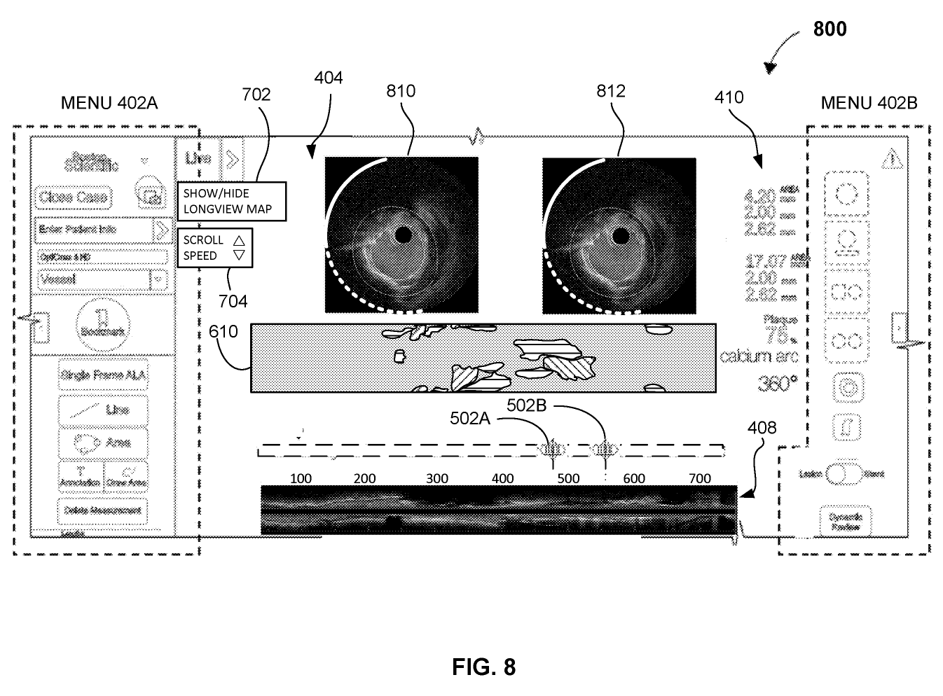

FIG. 8 illustrates an eleventh example GUI 800, which can be generated according to some embodiments of the present disclosure. For example, GUI 800 can be generated by IVUS image visualization system 300 as GUI 324 and displayed on display 304. With some embodiments, responsive to receiving the series of IVUS image frames 200A, processor 306 can execute instructions 316 to detect obstruction 116 and generate detected calcium reverb 322. Further, processor 306 can execute instructions 316 to generate GUI 800 comprising visual indications of the detected attenuated plaque 330.

Embodiments of GUI 800 include many of the features included in GUI 700 as described above, including a display of mapping 610. GUI 800 differs from GUI 700 in that GUI 800 includes a pair of slider bars, 502A and 502B, and a pair of cross-sectional images 810 and 812. In various embodiments, slider bar 502A can be moved and positioned independently of slider bar 502B. In various embodiments, image 810 is configured to display, in a cross-sectional image, the portion of the vessel provided in mapping 610 and in longview 408 that corresponds to the position of slider bar 502A, while image 812 is configured to display, in a cross-sectional image, the portion of the vessel depicted in mapping 610 and in longview 408 that corresponds to the position of slider bar 502B. By having individually controllable slider bars, a user such as a physician is able to compare images on a side by side basis that represents two different longitudinal locations along the length of the vessel depicted in mapping 610 and longview 408. Various advantages, such as estimating a length along the vessel between two different points of interest, and the ability to compare and contrast the statuses of different location along the longitudinal length of the vessel, are presented by the display of information that can be presented by GUI 800.

Although FIG. 8 illustrates slider bars 502A and 502B as one example of user-positionable longitudinal markers, it is to be appreciated that GUI 800 is not limited to slider bars. In various embodiments, each of the first and second user-positionable longitudinal markers may comprise a slider bar, a bookmark associated with a saved longitudinal position along a longitudinal axis of the longitudinal view, a draggable pin marker, or any other graphical element configured to be positioned along the longitudinal axis of the longitudinal view. In various embodiments, the first user-positionable longitudinal marker may comprise a different type of marker than the second user-positionable longitudinal marker. For example, in some embodiments the first user-positionable longitudinal marker may comprise a slider bar and the second user-positionable longitudinal marker may comprise a bookmark associated with a saved longitudinal position along the longitudinal axis. In each such embodiment, image 810 is configured to display, in a cross-sectional image, the portion of the vessel that corresponds to the position of the first user-positionable longitudinal marker, and image 812 is configured to display, in a cross-sectional image, the portion of the vessel that corresponds to the position of the second user-positionable longitudinal marker, the first user-positionable longitudinal marker being positionable independently of the second user-positionable longitudinal marker. In embodiments in which a user-positionable longitudinal marker comprises a bookmark, the bookmark may be generated, in response to a user input designating a longitudinal position along the longitudinal axis, at the designated longitudinal position, and may thereafter be recalled to drive the corresponding one of image 810 or image 812 to a cross-sectional image of the vessel at the saved longitudinal position.

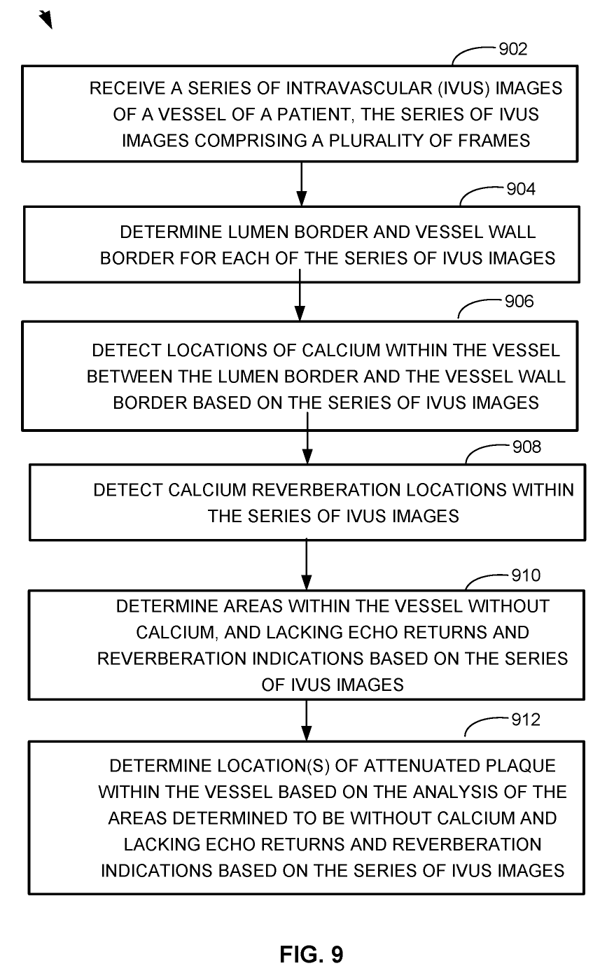

FIG. 9 illustrates a logic flow 900, according to some embodiments of the present disclosure. The logic flow 900 can be implemented by IVUS image visualization system 300 and will be described with reference to IVUS image visualization system 300 for clarity of presentation. However, it is noted that logic flow 900 could also be implemented by an IVUS guidance system different than IVUS image visualization system 300.

Logic flow 900 begins at block 902. At block 902, “receive a series of intravascular ultrasound (IVUS) images of a vessel of a patient,” a series of IVUS images captured via an IVUS catheter percutaneously inserted in a vessel of a patent can be received. For example, information elements comprising indications of the series of IVUS image frames 200A can be received from IVUS imaging system 118 where catheter 106 is (or was) percutaneously inserted into cardiac artery 102. The series of IVUS image frames 200A can comprise frames (e.g., IVUS image frames 202-1 to 202-n) representative of images captured while the catheter 106 is pulled back from the distal point 112 to the proximal point 114. Processor 306 can execute instructions 316 to receive information elements comprising indications of the series of IVUS image frames 200A from IVUS imaging system 118, or directly from catheter 106 as may be the case.

Continuing to block 904, “determine lumen border and vessel wall border for each of the series of IVUS images,” processor 306 can execute instructions 316, which may include a machine learning and/or a graphics processing application, to determine the location of the lumen border and the vessel wall border for each of the images included in the series of IVUS image frames.

Continuing to block 906, “detect locations of calcium within the vessel between the lumen border and the vessel wall border based on the series of IVUS images, “processor 306 can execute instructions 316 to detect (e.g., using machine learning and/or image processing) the location of calcium in the vessel based on the series of IVUS image frames.

Continuing to block 908, “detect calcium reverb locations within the series of IVUS images” calcium reverb can be detected from the series of IVUS images. For example, processor 306 can execute instructions 316 to detect (e.g., using machine learning and/or image processing) calcium reverb in the vessel based on the frames of series of IVUS image frames 200A. In various embodiments, the detected calcium reverb is filtered out of the images for each of the series of IVUS images using a filtering application, such as a graphics processing application.

Continuing to block 910, “determine area within the vessel without calcium and lacking echo returns and reverberations indications based on the image information included in each of the series of IVUS images.” For example, processor 306 can execute instructions 316 to determine, using a machine learning model and/or graphic processing, areas within the intra-border areas included between the lumen border and the vessel wall border that lack an echo return indication based on the image information included in each of the series of IVUS images. A lack of echo return may be indicated by low level of intensity for a signal return from an area with the intra-border area defined by the lumen border and the vessel wall border.

Continuing to block 912, “determine location(s) of attenuated plaque within the vessel based on the analysis of the areas determined to be without calcium and lacking echo returns and reverberation indications based on the information included in the IVUS images.” For example, processor 306 can execute instructions 316 the analyze the areas outside of the vessel wall border, and along one or more vectors extending into the areas outside of the vessel wall border in the IVUS image frame to determine where a lack of an echo response exists in these outside areas. A lack of echo response in an area outside of the vessel wall border is an indication that attenuated plaque has absorbed the ultrasound single transmitted along the vector and therefore indicates the presence of attenuated plaque for that portion of the vessel and at the location of the vector where no echo respond was received back. In various embodiments, the determination of the location of attenuated plaque includes mapping the location of the detected attenuated plaque based on the location along the vessel corresponding to the IVUS image frame where the attenuated plaque was detected, and in the orientation indicated by one or more of the vectors within the image where the lack of an echo from outside the vessel wall border was indicated.

With some embodiments, the processor 306 can execute the instructions 316 to identify areas of vulnerable plaque based on (1) determining whether the plaque burden (PB) is greater than or equal to 70 percent (%) and/or (2) determine whether the minimum lumen area (MLA) is less than or equal to 4 millimeters (mm). For example, areas where the PB is greater than or equal to 70 % and where the MLA is less than or equal to 4 mm can be flagged as potential areas of attenuated plaque. Further, with some embodiments, the processor 306 can execute the instructions 316 to identify areas of attenuated plaque vulnerable where the PB is greater than or equal to 70%, the MLA is less than or equal to 4 mm and the area has an arc length of the greater than 90 degrees and/or longer than 1 mm.

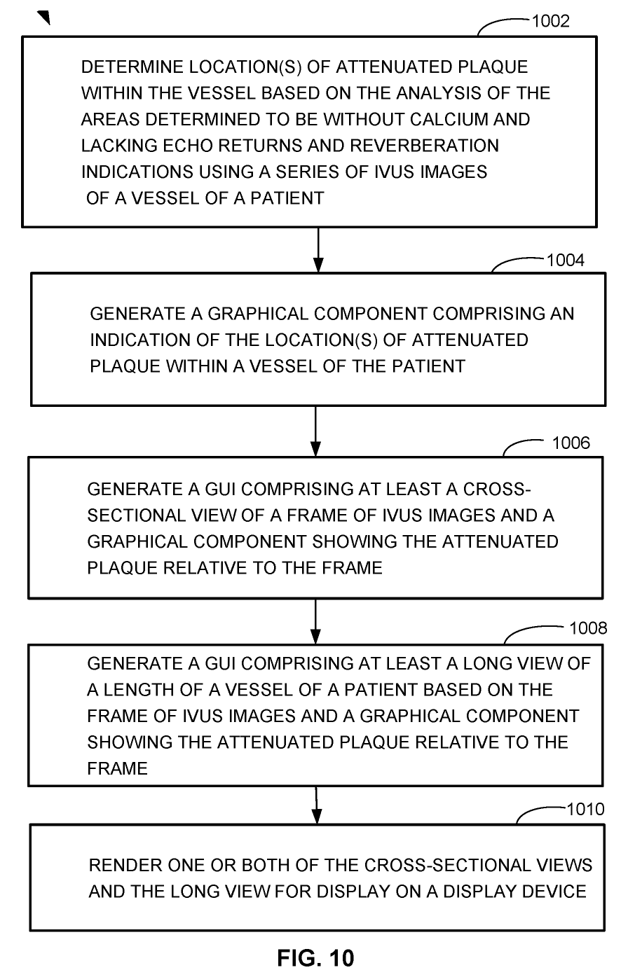

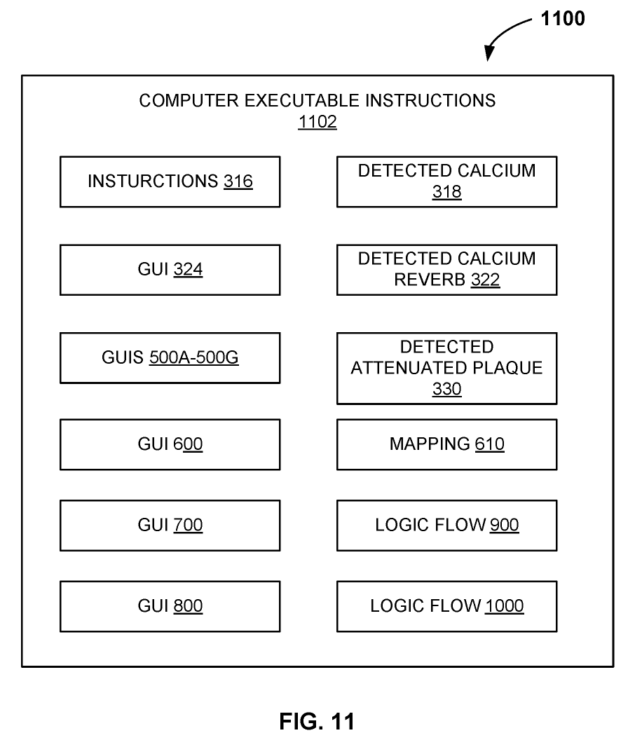

FIG. 10 illustrates a logic flow 1000, according to some embodiments of the present disclosure. The logic flow 1000 can be implemented by IVUS image visualization system 300 and will be described with reference to IVUS image visualization system 300 for clarity of presentation. However, it is noted that logic flow 1000 could also be implemented by an IVUS guidance system different than IVUS image visualization system 300.

Logic flow 1000 begins at block 1002. At block 1002, “receiving a series of intravascular ultrasound (IVUS) images of a vessel of a patient,” wherein a series of IVUS images captured via an IVUS catheter percutaneously inserted in a vessel of a patient can be received. For example, information elements comprising indications of the series of IVUS image frames 200A can be received from IVUS imaging system 118 where catheter 106 is (or was) percutaneously inserted into cardiac artery 102. The series of IVUS image frames 200A can comprise frames (e.g., IVUS image frames 202-1 to 202-n) representative of images captured while the catheter 106 is pulled back from the distal point 112 to the proximal point 114. Processor 306 can execute instructions 316 to receive information elements comprising indications of series of IVUS image frames 200A from IVUS imaging system 118, or directly from catheter 106 as may be the case.

At block 1004 logic flow 1000 includes, “detecting areas of attenuated plaque within the vessel based on the series of IVUS images.” For example, processor 306 can execute instructions 316 using any of the methods and techniques as described herein, and/or any variations and equivalents thereof, to determine areas of the IVUS images that indicate the presence of attenuated plaque within the vessel. Embodiments of detecting attenuated plaque within the vessel based on the series of IVUS images include use of the method as illustrated and described above with respect to FIG. 9 and logic flow 900. The analysis of the series of IVUS images may include analysis to determine areas of the images without calcium and lacking echo returns and calcium reverb indications using any of the tools, methods, and techniques described in the present disclosure. In various embodiments, detecting areas of attenuated plaque within the vessel may comprises: determining, for each image of the series of IVUS images, a lumen border of the vessel depicted in the image; determining, for each image of the series of IVUS images, a vessel border of the vessel depicted in the image; and analyzing, for each image of the series of IVUS images, an intra-border area to determine a change in an intensity of the IVUS image within the intra-border area and along a vector extending through the intra-border area to determine a presence of attenuated plaque, wherein the intra-border area is the area of the image between the vessel border and the lumen border.

At block 1006 logic flow 1000 includes, “generating a graphical component comprising an indication of attenuated plaque relative to at least one image of the series of IVUS images.” For example, processor 306 can execute instructions 316 to generate the graphical component(s) based on the detected attenuated plaque. In various embodiments, the graphical component comprising the indication of the attenuated plaque comprises an arc extending around at least a portion of a circumference of the portion of the vessel depicted in the cross-sectional view. In various embodiments, the graphical component further comprises an indication of a length of the arc extending around the at least the portion of the circumference of the cross-sectional view and an indication of a rotational positioning of the arc relative to the circumference. In various embodiments, processor 306 can execute instructions 316 to generate a longitudinal view of the series of IVUS images, the longitudinal view including indications of areas along the vessel where attenuated plaque was detected. In various embodiments, the longitudinal view comprises a slider, and wherein the position of the slider along a longitudinal axis of the longitudinal view corresponds to the at least one image of the series of IVUS images represented on the cross-sectional view.

In various embodiments, generating a graphical component includes processor 306 executing instructions 316 to generate a vessel profile view from the series of IVUS images. In various embodiments, generating the graphical component include processor 306 executing instructions to generate a mapping of the areas of detected attenuated plaque within the vessel, the mapping comprising a longitudinal view of the series of IVUS images and graphical depictions of locations within the portion of the vessel represented in the longitudinal view where attenuated plaque was detected. In various embodiments, the mapping comprises a view of the detected attenuated plaque and wherein a longitudinal axis of the vessel is represented on an x axis of the view and a circumference of the vessel is represented on a y axis of the view. In various embodiments, generating the mapping comprises generating the areas of the detected attenuated plaque and areas of detected calcium. In various embodiments, processor 306 can execute instructions 316 that include generating graphical components based on detected calcium and/or calcium reverb indications based on the analysis of the series of IVUS images.

At block 1008 logic flow 1000 includes, “generating a cross-sectional view of the at least one image of the series of IVUS images.” For example, processor 306 can execute instructions 316 to generate the graphical components based on detected attenuated plaque.

At block 1010 logic flow 1000 includes, “generating a graphical user interface (GUI) comprising the cross-sectional view and the graphical component comprising the indication of the attenuated plaque.” For example, processor 306 may execute instructions 316 to generate GUI 324 comprising the cross-section view and the graphical component comprising the indication of the attenuated plaque. In various embodiments, generating the GUI 324 comprises overlaying the graphical component onto the view of the at least one of the series of IVUS images. In various embodiments, generating the GUI includes generating a longitudinal view of the series of IVUS images. In various embodiments, the GUI comprises the cross-sectional view, the longitudinal view, and the graphical component. In various embodiments, the GUI comprises the cross-sectional view, the vessel profile view, and the graphical component. In various embodiments, the graphical component further comprises an indication of the mapping. In various embodiments, generating the GUI comprises overlaying indications of a lumen border and a vessel border on the cross-sectional view. In various embodiments, generating the GUI comprises positioning the mapping offset from and aligned with a longitudinal view so that one or more locations within the vessel where attenuated plaque was detected align with corresponding portions of the image in the longitudinal view of the vessel.

At block 1012 logic flow 1000 includes, “rendering the GUI for display on a display.” For example, processor 306 may execute instructions 306 to cause GUI 324 to be rendered for display on a display, such as display 304.

FIG. 11 illustrates computer-readable storage medium 1100. Computer-readable storage medium 1100 may comprise any non-transitory computer-readable storage medium or machine-readable storage medium, such as an optical, magnetic or semiconductor storage medium. In various embodiments, computer-readable storage medium 1100 may comprise an article of manufacture. In some embodiments, computer-readable storage medium 1100 may store computer executable instructions 1102 with which circuitry (e.g., processor 124, processor 306, IVUS imaging system acquisition circuitry 314, and the like) can execute. For example, computer executable instructions 1102 can include instructions to implement operations described with respect to instructions 316, detected calcium 318, detected calcium reverb 322, detected attenuated plaque 330, GUI 324, GUI 500A, GUI 500B, GUI 500C, GUI 500D, GUI 500E, GUI 500F, GUI 500G, mapping 610, GUI 600, GUI 700, GUI 800, logic flow 900, and/or logic flow 1000. Examples of computer-readable storage medium 1100 or machine-readable storage medium may include any tangible media capable of storing electronic data, including volatile memory or non-volatile memory, removable or non-removable memory, erasable or non-erasable memory, writeable or re-writeable memory, and so forth. Examples of computer executable instructions 1102 may include any suitable type of code, such as source code, compiled code, interpreted code, executable code, static code, dynamic code, object-oriented code, visual code, and the like.

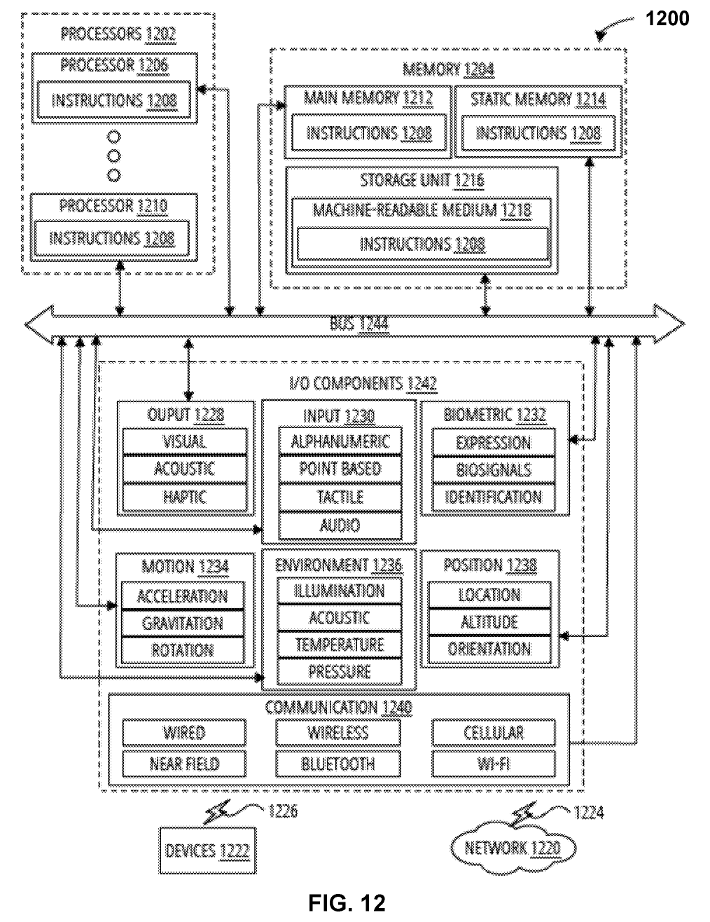

FIG. 12 illustrates a diagrammatic representation of a machine 1200 in the form of a computer system within which a set of instructions may be executed for causing the machine to perform any one or more of the methodologies discussed herein. More specifically, FIG. 12 shows a diagrammatic representation of the machine 1200 in the example form of a computer system, within which instructions 1208 (e.g., software, a program, an application, an applet, an app, or other executable code) for causing the machine 1200 to perform any one or more of the methodologies discussed herein may be executed. For example, the instructions 1208 may cause the machine 1200 to execute logic flow 900 of FIG. 9, logic flow 1000 of FIG. 10, instructions 316 of FIG. 3. More generally, the instructions 1208 may cause the machine 1200 to generate GUIs with functionality and behavior as described herein during a pre-PCI, peri-PCI, or post-PCI using IVUS. It is noted that the present disclosure provides specific and discrete implementations of GUI representations and behavior that is a significant improvement over the prior art. In particular, the present disclosure provides an improvement to computing technology in that GUIs provide greater visibility and navigation of IVUS images including notation of locations of calcium reverb.

The instructions 1208 transform the general, non-programmed machine 1200 into a particular machine 1200 programmed to carry out the described and illustrated functions in a specific manner. In alternative embodiments, the machine 1200 operates as a standalone device or may be coupled (e.g., networked) to other machines. In a networked deployment, the machine 1200 may operate in the capacity of a server machine or a client machine in a server-client network environment, or as a peer machine in a peer-to-peer (or distributed) network environment. The machine 1200 may comprise, but not be limited to, a server computer, a client computer, a personal computer (PC), a tablet computer, a laptop computer, a netbook, a set-top box (STB), a PDA, an entertainment media system, a cellular telephone, a smart phone, a mobile device, a wearable device (e.g., a smart watch), a smart home device (e.g., a smart appliance), other smart devices, a web appliance, a network router, a network switch, a network bridge, or any machine capable of executing the instructions 1208, sequentially or otherwise, that specify actions to be taken by the machine 1200. Further, while only a single machine 1200 is illustrated, the term “machine” shall also be taken to include a collection of machines 1200 that individually or jointly execute the instructions 1208 to perform any one or more of the methodologies discussed herein.

The machine 1200 may include processors 1202, memory 1204, and I/O components 1242, which may be configured to communicate with each other such as via a bus 1244. In an example embodiment, the processors 1202 (e.g., a Central Processing Unit (CPU), a Reduced Instruction Set Computing (RISC) processor, a Complex Instruction Set Computing (CISC) processor, a Graphics Processing Unit (GPU), a Digital Signal Processor (DSP), an ASIC, a Radio-Frequency Integrated Circuit (RFIC), another processor, or any suitable combination thereof) may include, for example, a processor 1206 and a processor 1210 that may execute the instructions 1208. The term “processor” is intended to include multi-core processors that may comprise two or more independent processors (sometimes referred to as “cores”) that may execute instructions contemporaneously. Although FIG. 12 shows multiple processors 1202, the machine 1200 may include a single processor with a single core, a single processor with multiple cores (e.g., a multi-core processor), multiple processors with a single core, multiple processors with multiples cores, or any combination thereof.

The memory 1204 may include a main memory 1212, a static memory 1214, and a storage unit 1216, both accessible to the processors 1202 such as via the bus 1244. The main memory 1204, the static memory 1214, and storage unit 1216 store the instructions 1208 embodying any one or more of the methodologies or functions described herein. The instructions 1208 may also reside, completely or partially, within the main memory 1212, within the static memory 1214, within machine-readable medium 1218 within the storage unit 1216, within at least one of the processors 1202 (e.g., within the processor’s cache memory), or any suitable combination thereof, during execution thereof by the machine 1200.

The I/O components 1242 may include a wide variety of components to receive input, provide output, produce output, transmit information, exchange information, capture measurements, and so on. The specific I/O components 1242 that are included in a particular machine will depend on the type of machine. For example, portable machines such as mobile phones will likely include a touch input device or other such input mechanisms, while a headless server machine will likely not include such a touch input device. It will be appreciated that the I/O components 1242 may include many other components that are not shown in FIG. 12. The I/O components 1242 are grouped according to functionality merely for simplifying the following discussion and the grouping is in no way limiting. In various example embodiments, the I/O components 1242 may include output components 1228 and input components 1230. The output components 1228 may include visual components (e.g., a display such as a plasma display panel (PDP), a light emitting diode (LED) display, a liquid crystal display (LCD), a projector, or a cathode ray tube (CRT)), acoustic components (e.g., speakers), haptic components (e.g., a vibratory motor, resistance mechanisms), other signal generators, and so forth. The input components 1230 may include alphanumeric input components (e.g., a keyboard, a touch screen configured to receive alphanumeric input, a photo-optical keyboard, or other alphanumeric input components), point-based input components (e.g., a mouse, a touchpad, a trackball, a joystick, a motion sensor, or another pointing instrument), tactile input components (e.g., a physical button, a touch screen that provides location and/or force of touches or touch gestures, or other tactile input components), audio input components (e.g., a microphone), and the like.

In further example embodiments, the I/O components 1242 may include biometric components 1232, motion components 1234, environmental components 1236, or position components 1238, among a wide array of other components. For example, the biometric components 1232 may include components to detect expressions (e.g., hand expressions, facial expressions, vocal expressions, body gestures, or eye tracking), measure bio signals (e.g., blood pressure, heart rate, body temperature, perspiration, or brain waves), identify a person (e.g., voice identification, retinal identification, facial identification, fingerprint identification, or electroencephalogram-based identification), and the like. The motion components 1234 may include acceleration sensor components (e.g., accelerometer), gravitation sensor components, rotation sensor components (e.g., gyroscope), and so forth. The environmental components 1236 may include, for example, illumination sensor components (e.g., photometer), temperature sensor components (e.g., one or more thermometers that detect ambient temperature), humidity sensor components, pressure sensor components (e.g., barometer), acoustic sensor components (e.g., one or more microphones that detect background noise), proximity sensor components (e.g., infrared sensors that detect nearby objects), gas sensors (e.g., gas detection sensors to detection concentrations of hazardous gases for safety or to measure pollutants in the atmosphere), or other components that may provide indications, measurements, or signals corresponding to a surrounding physical environment. The position components 1238 may include location sensor components (e.g., a GPS receiver component), altitude sensor components (e.g., altimeters or barometers that detect air pressure from which altitude may be derived), orientation sensor components (e.g., magnetometers), and the like.

Communication may be implemented using a wide variety of technologies. The I/O components 1242 may include communication components 1240 operable to couple the machine 1200 to a network 1220 or devices 1222 via a coupling 1224 and a coupling 1226, respectively. For example, the communication components 1240 may include a network interface component or another suitable device to interface with the network 1220. In further examples, the communication components 1240 may include wired communication components, wireless communication components, cellular communication components, Near Field Communication (NFC) components, Bluetooth^{®} components (e.g., Bluetooth^{®} Low Energy), Wi-Fi^{®} components, and other communication components to provide communication via other modalities. The devices 1222 may be another machine or any of a wide variety of peripheral devices (e.g., a peripheral device coupled via a USB).

Moreover, the communication components 1240 may detect identifiers or include components operable to detect identifiers. For example, the communication components 1240 may include Radio Frequency Identification (RFID) tag reader components, NFC smart tag detection components, optical reader components (e.g., an optical sensor to detect one-dimensional bar codes such as Universal Product Code (UPC) bar code, multi-dimensional bar codes such as Quick Response (QR) code, Aztec code, Data Matrix, Dataglyph, MaxiCode, PDF417, Ultra Code, UCC RSS-2D bar code, and other optical codes), or acoustic detection components (e.g., microphones to identify tagged audio signals). In addition, a variety of information may be derived via the communication components 1240, such as location via Internet Protocol (IP) geolocation, location via Wi-Fi® signal triangulation, location via detecting an NFC beacon signal that may indicate a particular location, and so forth.

The various memories (i.e., memory 1204, main memory 1212, static memory 1214, and/or memory of the processors 1202) and/or storage unit 1216 may store one or more sets of instructions and data structures (e.g., software) embodying or utilized by any one or more of the methodologies or functions described herein. These instructions (e.g., the instructions 1208), when executed by processors 1202, cause various operations to implement the disclosed embodiments.

As used herein, the terms “machine-storage medium,” “device-storage medium,” “computer-storage medium” mean the same thing and may be used interchangeably in this disclosure. The terms refer to a single or multiple storage devices and/or media (e.g., a centralized or distributed database, and/or associated caches and servers) that store executable instructions and/or data. The terms shall accordingly be taken to include, but not be limited to, solid-state memories, and optical and magnetic media, including memory internal or external to processors. Specific examples of machine-storage media, computer-storage media and/or device-storage media include non-volatile memory, including by way of example semiconductor memory devices, e.g., erasable programmable read-only memory (EPROM), electrically erasable programmable read-only memory (EEPROM), FPGA, and flash memory devices; magnetic disks such as internal hard disks and removable disks; magneto-optical disks; and CD-ROM and DVD-ROM disks. The terms “machine-storage media,” “computer-storage media,” and “device-storage media” specifically exclude carrier waves, modulated data signals, and other such media, at least some of which are covered under the term “signal medium” discussed below.

In various example embodiments, one or more portions of the network 1220 may be an ad hoc network, an intranet, an extranet, a VPN, a LAN, a WLAN, a WAN, a WWAN, a MAN, the Internet, a portion of the Internet, a portion of the PSTN, a plain old telephone service (POTS) network, a cellular telephone network, a wireless network, a Wi-Fi® network, another type of network, or a combination of two or more such networks. For example, the network 1220 or a portion of the network 1220 may include a wireless or cellular network, and the coupling 1224 may be a Code Division Multiple Access (CDMA) connection, a Global System for Mobile communications (GSM) connection, or another type of cellular or wireless coupling. In this example, the coupling 1224 may implement any of a variety of types of data transfer technology, such as Single Carrier Radio Transmission Technology (1xRTT), Evolution-Data Optimized (EVDO) technology, General Packet Radio Service (GPRS) technology, Enhanced Data rates for GSM Evolution (EDGE) technology, third Generation Partnership Project (3GPP) including 3G, fourth generation wireless (4G) networks, Universal Mobile Telecommunications System (UMTS), High Speed Packet Access (HSPA), Worldwide Interoperability for Microwave Access (WiMAX), Long Term Evolution (LTE) standard, others defined by various standard-setting organizations, other long range protocols, or other data transfer technology.

The instructions 1208 may be transmitted or received over the network 1220 using a transmission medium via a network interface device (e.g., a network interface component included in the communication components 1240) and utilizing any one of several well-known transfer protocols (e.g., hypertext transfer protocol (HTTP)). Similarly, the instructions 1208 may be transmitted or received using a transmission medium via the coupling 1226 (e.g., a peer-to-peer coupling) to the devices 1222. The terms “transmission medium” and “signal medium” mean the same thing and may be used interchangeably in this disclosure. The terms “transmission medium” and “signal medium” shall be taken to include any intangible medium that is capable of storing, encoding, or carrying the instructions 1208 for execution by the machine 1200, and includes digital or analog communications signals or other intangible media to facilitate communication of such software. Hence, the terms “transmission medium” and “signal medium” shall be taken to include any form of modulated data signal, carrier wave, and so forth. The term “modulated data signal” means a signal that has one or more of its characteristics set or changed in such a matter as to encode information in the signal.

Terms used herein should be accorded their ordinary meaning in the relevant arts, or the meaning indicated by their use in context, but if an express definition is provided, that meaning controls.

Herein, references to "one embodiment" or "an embodiment" do not necessarily refer to the same embodiment, although they may. Unless the context clearly requires otherwise, throughout the description and the claims, the words "comprise," "comprising," and the like are to be construed in an inclusive sense as opposed to an exclusive or exhaustive sense; that is to say, in the sense of "including, but not limited to." Words using the singular or plural number also include the plural or singular number respectively, unless expressly limited to one or multiple ones. Additionally, the words "herein," "above," "below" and words of similar import, when used in this application, refer to this application as a whole and not to any portions of this application. When the claims use the word "or" in reference to a list of two or more items, that word covers all the following interpretations of the word: any of the items in the list, all the items in the list and any combination of the items in the list, unless expressly limited to one or the other. Any terms not expressly defined herein have their conventional meaning as commonly understood by those having skill in the relevant art(s).

Terms used herein should be accorded their ordinary meaning in the relevant arts, or the meaning indicated by their use in context, but if an express definition is provided, that meaning controls.

What is claimed is:

1. A method, comprising:
receiving a series of intravascular ultrasound (IVUS) images of a vessel of a patient;
generating a longitudinal view of the vessel based on the series of IVUS images;
generating a first cross-sectional view of a first one of the series of IVUS images and a second cross-sectional view of a second one of the series of IVUS images;
generating a graphical user interface (GUI) comprising the longitudinal view, the first cross-sectional view, the second cross-sectional view, a first user-positionable longitudinal marker, and a second user-positionable longitudinal marker, wherein the first longitudinal marker and the second longitudinal marker are each positionable along a longitudinal axis of the longitudinal view, wherein the first longitudinal marker is positionable independently of the second longitudinal marker, wherein the first cross-sectional view is linked to the first longitudinal marker, and wherein the second cross-sectional view is linked to the second longitudinal marker;
updating the first cross-sectional view, responsive to a change in a position of the first longitudinal marker along the longitudinal axis, to depict a cross-sectional image of the vessel corresponding to the position of the first longitudinal marker;
updating the second cross-sectional view, responsive to a change in a position of the second longitudinal marker along the longitudinal axis, to depict a cross-sectional image of the vessel corresponding to the position of the second longitudinal marker; and
rendering the GUI for display on a display.

2. The method of claim 1, wherein at least one of the first user-positionable longitudinal marker or the second user-positionable longitudinal marker comprises a slider bar positionable along the longitudinal axis of the longitudinal view by user input.

3. The method of claim 1, wherein at least one of the first user-positionable longitudinal marker or the second user-positionable longitudinal marker comprises a bookmark associated with a saved position along the longitudinal axis of the longitudinal view.

4. The method of claim 3, further comprising:
receiving a user input designating a position along the longitudinal axis of the longitudinal view; and
generating the bookmark at the designated position responsive to the user input.

5. The method of claim 1, wherein at least one of the first user-positionable longitudinal marker or the second user-positionable longitudinal marker comprises a draggable pin marker.

6. The method of claim 1, wherein the first user-positionable longitudinal marker comprises a slider bar and the second user-positionable longitudinal marker comprises a bookmark associated with a saved position along the longitudinal axis of the longitudinal view.

7. The method of claim 1, further comprising:
detecting areas of attenuated plaque within the vessel based on the series of IVUS images; and
generating a graphical component comprising an indication of the attenuated plaque,
wherein the GUI comprises the graphical component.

8. The method of claim 7, wherein the graphical component comprises a first arc extending around at least a portion of a circumference of the vessel depicted in the first cross-sectional view and a second arc extending around at least a portion of a circumference of the vessel depicted in the second cross-sectional view.

9. The method of claim 1, wherein the first cross-sectional view and the second cross-sectional view are displayed side by side within the GUI.

10. The method of claim 1, further comprising determining a longitudinal distance along the vessel between the position of the first user-positionable longitudinal marker and the position of the second user-positionable longitudinal marker, wherein the GUI further comprises an indication of the longitudinal distance.

11. The method of claim 1, further comprising:
generating a mapping comprising graphical depictions of locations within the vessel where attenuated plaque was detected, the mapping comprising a long view of the series of IVUS images,
wherein a longitudinal axis of the vessel is represented on an x axis of the mapping and a circumference of the vessel is represented on a y axis of the mapping, and
wherein the GUI further comprises the mapping positioned offset from and aligned with the longitudinal view so that one or more locations within the vessel where attenuated plaque was detected align with corresponding portions of the longitudinal view.

12. The method of claim 1, further comprising:
generating a graphical display representing a portion of an anatomical structure within which the vessel resides; and
generating a position indicator within the graphical display, the position indicator indicating, within the anatomical structure, a location of the vessel corresponding to one of the position of the first user-positionable longitudinal marker or the position of the second user-positionable longitudinal marker,
wherein the GUI further comprises the graphical display and the position indicator.

13. The method of claim 12, wherein the position indicator comprises a dashed line.

14. The method of claim 12, wherein the GUI further comprises a toggle input operable to selectively show or hide the graphical display within the GUI.

15. The method of claim 12, further comprising:
receiving an input from a scroll speed control of the GUI; and
automatically advancing the position of the first user-positionable longitudinal marker along the longitudinal axis at a rate corresponding to the input received from the scroll speed control,
wherein the first cross-sectional view and the position indicator are updated as the position of the first user-positionable longitudinal marker advances.

16. The method of claim 15, wherein automatically advancing the position of the first user-positionable longitudinal marker comprises advancing the position of the first user-positionable longitudinal marker independently of the position of the second user-positionable longitudinal marker.

17. The method of claim 12, wherein the anatomical structure comprises a portion of cardiac vasculature, and wherein the graphical display comprises a representation of a plurality of branches of the cardiac vasculature.

18. An apparatus for visualizing intravascular ultrasound images, comprising:
a processor arranged to be coupled to a non-transitory memory storage device, the non-transitory memory storage device comprising computer readable instructions that, when executed by the processor, cause the apparatus to:
receive a series of intravascular ultrasound (IVUS) images of a vessel of a patient;
generate a longitudinal view of the vessel based on the series of IVUS images;
generate a first cross-sectional view of a first one of the series of IVUS images and a second cross-sectional view of a second one of the series of IVUS images;
generate a graphical user interface (GUI) comprising the longitudinal view, the first cross-sectional view, the second cross-sectional view, a first user-positionable longitudinal marker, and a second user-positionable longitudinal marker, wherein the first longitudinal marker and the second longitudinal marker are each positionable along a longitudinal axis of the longitudinal view, wherein the first longitudinal marker is positionable independently of the second longitudinal marker, wherein the first cross-sectional view is linked to the first longitudinal marker, and wherein the second cross-sectional view is linked to the second longitudinal marker;
update the first cross-sectional view, responsive to a change in a position of the first longitudinal marker along the longitudinal axis, to depict a cross-sectional image of the vessel corresponding to the position of the first longitudinal marker;
update the second cross-sectional view, responsive to a change in a position of the second longitudinal marker along the longitudinal axis, to depict a cross-sectional image of the vessel corresponding to the position of the second longitudinal marker; and
render the GUI for display on a display.

19. The apparatus of claim 18, wherein at least one of the first user-positionable longitudinal marker or the second user-positionable longitudinal marker comprises a slider bar, a bookmark associated with a saved position along the longitudinal axis of the longitudinal view, or a draggable pin marker.

20. A non-transitory computer-readable storage device comprising instructions executable by a processor of a computing device, wherein the instructions, when executed by the processor, cause the computing device to:
receive a series of intravascular ultrasound (IVUS) images of a vessel of a patient;
generate a longitudinal view of the vessel based on the series of IVUS images;
generate a first cross-sectional view of a first one of the series of IVUS images and a second cross-sectional view of a second one of the series of IVUS images;
generate a graphical user interface (GUI) comprising the longitudinal view, the first cross-sectional view, the second cross-sectional view, a first user-positionable longitudinal marker, and a second user-positionable longitudinal marker, wherein the first longitudinal marker and the second longitudinal marker are each positionable along a longitudinal axis of the longitudinal view, wherein the first longitudinal marker is positionable independently of the second longitudinal marker, wherein the first cross-sectional view is linked to the first longitudinal marker, and wherein the second cross-sectional view is linked to the second longitudinal marker;
update the first cross-sectional view, responsive to a change in a position of the first longitudinal marker along the longitudinal axis, to depict a cross-sectional image of the vessel corresponding to the position of the first longitudinal marker;
update the second cross-sectional view, responsive to a change in a position of the second longitudinal marker along the longitudinal axis, to depict a cross-sectional image of the vessel corresponding to the position of the second longitudinal marker; and
render the graphical user interface for display on a display.
